(19) United States
(12) Patent Application Publication
(10) Pub. No.: US 2024/0054254 A1
(43) Pub. Date: Feb. 15, 2024

(54) SYSTEMS AND METHODS FOR USER SELECTION OF PARAMETERS TO APPROXIMATE DESIRED PROPERTIES OF LIGHT SCATTERING

(71) Applicant: BASF SE, Ludwigshafen (DE)

(72) Inventors: Keith Task, Beachwood, OH (US); Rupa Hiremath Darji, Tarrytown, NY (US); Michael David Burke, Tarrytown, NY (US); Liangliang Qu, Tarrytown, NY (US)

(21) Appl. No.: 18/278,535

(22) PCT Filed: Mar. 1, 2022
(86) PCT No.: PCT/IB2022/051796

§ 371 Date: Aug. 23, 2023

Related U.S. Application Data
(60) Provisional application No. 63/200,345, filed on Mar. 2, 2021.

(51) Int. Cl. G06F 30/12; G06F 30/25; B82Y 35/00

(52) U.S. Cl. CPC G06F 30/12; G06F 30/25; B82Y 35/00; G06F 2111/16

(57) ABSTRACT

The following relates generally to user selection of parameters to approximate desired properties of light scattering. More specifically, in some embodiments, a graphical user interface (GUI) is provided. In some embodiments, in a forward configuration, the GUI accepts an input parameter that is a physical property or optical property of a nanoparticle material (e.g., nanoparticle size, shape, etc.), and outputs an optical effect of the nanoparticle material (e.g., a color, a graph of a reflectance fraction vs. wavelength, etc.); whereas, in an inverse configuration, the GUI accepts the optical effect as the input, and outputs a physical property or optical property of the nanoparticle material.

### BACKGROUND

Structural color relies on the physical properties of a material to produce color. These physical properties give rise to interference among scattered light waves, which gives the scattered light a particular color. This makes structural color different from other types of color that rely on the chemical properties of the material.

Although structural color is common in nature, it has been difficult to reproduce by conventional techniques. Among other reasons, there is an extremely large number of configurations of physical properties that can affect the way that light scatters from a material. Moreover, when the light scatters from the surface, the interference effects can be hard to predict because of the complex ways that the light interacts with the material and with itself. These problems make it difficult to identify a specific set (or sets) of properties that will give rise to a specific desired structural color.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one aspect, there is a computer-implemented method. The method may include: displaying, by a processor, a plurality of modeling objectives; receiving, at the processor, a selected objective of the plurality of modeling objectives; displaying, by the processor, available types of models; receiving, at the processor, a selected type of model of the available types of models; displaying, by the processor, a request for at least one simulation input based on the selected type of model; receiving, at the processor, an entry for the at least one simulation input; displaying, by the processor, a request for at least one physical parameter; receiving, at the processor, an entry for the at least one physical parameter; and displaying, by the processor, an output parameter based on: (i) the selected objective, (ii) the selected type of model, (iii) the entry for the at least one simulation input, and (iv) the entry of the at least one physical parameter.

In another aspect, there is a computer system including one or more processors. The one or more processors may be configured to: display a plurality of modeling objectives; receive a selected objective of the plurality of modeling objectives; display available types of models; receive a selected type of model of the available types of models; display a request for at least one simulation input based on the selected type of model; receive an entry for the at least one simulation input; display a request for at least one physical parameter; receive an entry for the at least one physical parameter; and display an output parameter based on: (i) the selected objective, (ii) the selected type of model, (iii) the entry for the at least one simulation input, and (iv) the entry of the at least one physical parameter.

In yet another aspect, there is a computer-implemented method. The method may include: displaying, by a processor, a request for an optical property objective; receiving, at the processor, the optical property objective; displaying, by the processor, a request for a decision variable; receiving, at the processor, the decision variable; displaying, by the processor, a request for an optimization parameter; receiving, at the processor, the optimization parameter; and displaying, by the processor, and based on the optical property objective, the decision variable, and the optimization parameter: (i) an optimized reflectance curve, optimized transmittance curve, or optimized absorption curve or (ii) an optimized material property.

In yet another aspect, there is a computer system including one or more processors. The one or more processors may be configured to: display a request for an optical property objective; receive the optical property objective; display a request for a decision variable; receive the decision variable; display a request for an optimization parameter; receive the optimization parameter; and display based on the optical property objective, the decision variable, and the optimization parameter: (i) an optimized reflectance curve, or (ii) an optimized material property.

### BRIEF DESCRIPTION OF THE DRAWINGS

FIG. 1A is a diagram illustrating optical processes that contribute to non-structural color of a material.

FIG. 1B is a diagram illustrating optical processes that contribute to structural color of a material.

FIG. 2A is a diagram illustrating geometric parameters for determining the scattering of constructively, and destructively, interfering light.

FIG. 2B is a diagram depicting an array of three nanoparticles each scattering light waves demonstrative constructive and destructive interference.

FIG. 3A is a scanning electron microscopy (SEM) image of a microsphere having a direct, ordered structure.

FIG. 3B is a scanning electron microscopy (SEM) image of a microsphere having an inverse, ordered structure.

FIG. 3C is a scanning electron microscopy (SEM) image of a microsphere having a direct, disordered structure.

FIG. 3D is a scanning electron microscopy (SEM) image of a microsphere having an inverse, disordered structure.

FIG. 4A is a diagram that illustrates a simulated light scattering model according to a stochastic model, such as a Monte Carlo model.

FIG. 4B is a diagram that illustrates a bulk scattering model wherein incident light is incident on, and propagates into, a bulk material.

FIG. 5 illustrates an exemplary environment suitable for performing simulation(s) as described herein.

FIG. 6 illustrates an example of applying the environment of FIG. 5 to generate a reflectance curve.

FIG. 7 is a block diagram of an environment with a parameter refinement process that accepts, as input, target values for the outputs of the environment of FIG. 5.

FIG. 8 illustrates an exemplary system 800 suitable for practicing the simulation of structural color, and the parameter optimization techniques.

FIG. 9 illustrates a method suitable for selecting parameters of an array of nanoparticles.

FIG. 10 illustrates exemplary a method suitable for tuning parameters of an array of nanoparticles.

FIG. 11 depicts an illustrative computer system architecture that may be used to perform simulations of structural color, and perform optimizations of parameters of nanoparticles.

FIG. 12 illustrates an example system diagram for providing a GUI.

FIG. 13 illustrates an example method of a forward configuration aspect of the GUI.

FIG. 14A illustrates an example overall modeling options screen.

FIG. 14B illustrates an example Monte Carlo Simulation inputs screen.

FIG. 15 illustrates an example screen for entering physical parameters.

FIG. 16A illustrates an example screen for entering at least one matrix material refractive index property.

FIG. 16B illustrates an example screen for entering properties of matrix inclusions.

FIG. 17A illustrates an example display of an output parameter in the forward configuration of the GUI; in particular, FIG. 17A illustrates an example graph of a reflectance vs. wavelength.

FIG. 17B illustrates an example display of an output parameter in the forward configuration of the GUI; in particular, FIG. 17B illustrates an example graph of an absorption vs. wavelength.

FIG. 18 illustrates an example method of an inverse configuration aspect of the GUI.

FIG. 19 illustrates an example of a screen of the GUI in the inverse configuration.

FIG. 20 illustrates an example of a screen of the GUI in the inverse configuration, including an optimized reflectance curve.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

Embodiments described herein relate to techniques for determining optical properties of a nanoparticle or nanoparticle array and further to determining one or more structural properties of an array of nanoparticles that result in desired optical properties for a structural color of the array. The nanoparticle array may include, for example, an arrangement of nano-scale particles, a part of a single microsphere (a sphere measured at a micro-scale) made up of nanoparticles, or a bulk material including a plurality of microspheres. The techniques described herein may be used, for example, to produce the materials described in International Patent Application Publication No. WO 2019/051357, entitled “Microspheres Comprising Polydisperse Polymer Nanospheres and Porous Metal Oxide Microspheres” and filed on Sep. 10, 2018, and in International Patent Application Publication No. WO 2019/051353, entitled “Porous Metal Oxide Microspheres” and filed on Sep. 10, 2018. The contents of the aforementioned applications are incorporated by reference herein.

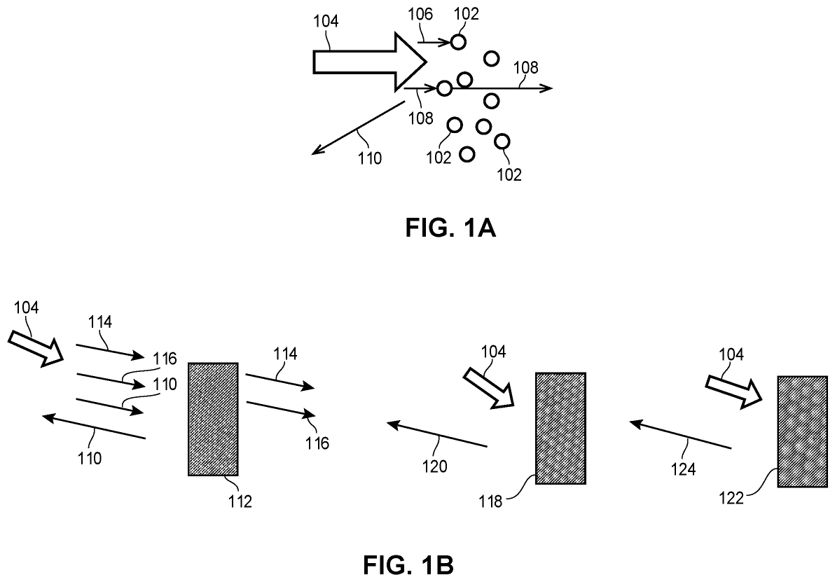

FIG. 1A is a diagram illustrating optical processes that contribute to non-structural color of a material, while FIG. 1B is a diagram illustrating optical processes that contribute to structural color of a material. As shown in FIG. 1A, a surface, liquid, aerosol, or other material may include a plurality of molecules 102. When incident white light 104, having a broadband of wavelengths, is directed at the molecules 102, some of the wavelengths of the white light 104 are absorbed, other wavelengths are scattered, and yet other wavelengths are transmitted by the plurality of molecules 102. The chemical properties of the molecules 102 determine which wavelengths of light are absorbed, which are scattered, and which are transmitted. For instance, in the example of FIG. 1A, the molecules 102 may absorb red light 106, transmit green light 108, and reflect or scatter blue light 110. Consequently, when a person looks at an object made up of the molecules 102, the object appears blue from the reflection of the blue light 110, or may appear green from the other side of the molecules 102 due to the transmission of the green light 108.

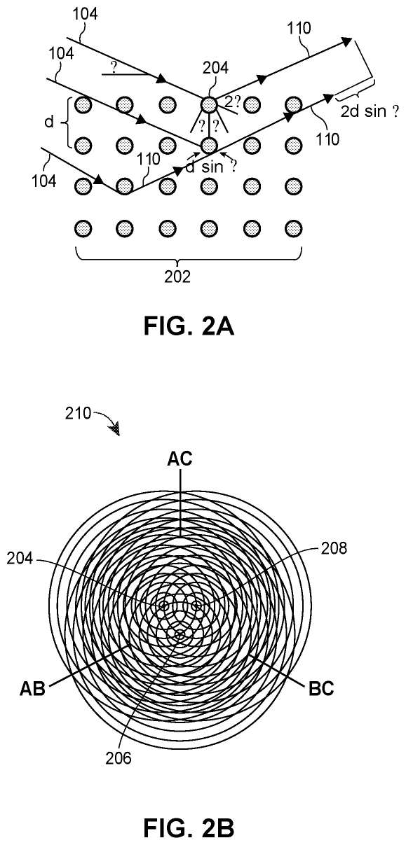

Structural color, as illustrated in FIG. 1B, is different from the chemically-induced color illustrated in FIG. 1A. Structural color can be caused by structures and geometries on the scale of the wavelengths of optical light (e.g., 400 to 800 nm). For example, structural color may be affected by the physical configuration of an array of nanoparticles. FIG. 1B depicts three nanoparticle arrays: a first array of nanoparticles 112, a second array of nanoparticles 118, and a third array of nanoparticles 122, with each array of nanoparticles having different physical properties. The physical properties of the first array of nanoparticles 112 cause some incident white light 104 to be transmitted (e.g., the red light 114 and the green light 116), while the blue light 110 is scattered. As described in more detail in connection with FIGS. 2A and 2B below, the scattered blue light 110 constructively interferes with itself, giving rise to a particular hue or shade of blue. By varying the physical properties of the first array of nanoparticles 112, the hue or shade can be altered.

The second array of nanoparticles 118 of FIG. 1B has a larger nanoparticle size as compared to the first array of nanoparticles 112. Consequently, the second array of nanoparticles 118 scatters green light 120 that constructively interferes with itself, instead of scattering the blue light 110. Similarly, the third array of nanoparticles 122, has an even larger nanoparticle size than the second array of nanoparticles 118, and the third array of nanoparticles scatters constructively-interfering red light 124.

FIG. 2A is a diagram illustrating geometric parameters for determining the scattering of constructively, and destructively, interfering light. FIG. 2A depicts an array of nanoparticles 202, with white light 104 being incident on a first nanoparticle 204 at an angle of incidence 0. The incident white light 104 is scattered by the first nanoparticle 204 and is reflected away from the array of nanoparticles 202 at a scattering angle that is equal to the angle of incidence 0. Further, the incident white light 104 may be scattered from other nanoparticles of the array of nanoparticles 202, either at a surface of the array of nanoparticles or within the array at a different layer of nanoparticles than the first nanoparticle 204.

FIG. 2B depicts an array of three nanoparticles each scattering light waves isotropically in the plane of the page. The illustration of FIG. 2B demonstrates constructive and destructive interference of scattered light from the nanoparticles. Light is incident on the first nanoparticle 204, a second nanoparticle 206, and a third nanoparticle 208. The light is scattered by the first, second, and third nanoparticles 204, 206, and 208. The scattered light waves are illustrated by the concentric rings of FIG. 2B, which each ring having a center at one of the first, second, or third nanoparticle 204, 206, or 208. The resulting aggregated scattered light gives rise to an interference pattern 210. The light waves constructively and destructively interfere at locations where two or more of the light waves are spatially overlapped. For example, the light scattered by the first and second nanoparticles 204 and 206 constructively interferes along the axis AB, light scattered by the second and third nanoparticles 206 and 208 constructively interferes along the axis BC, and light scattered by the first and third nanoparticles 204 and 208 constructively interferes along the axis AC. There are axes and locations where all scattered light from the three nanoparticles interferes constructively as well. Further, between the axes AB, BC, and AC, light scattered by the three nanoparticles 204, 206, and 208 includes regions of destructive interference, and may include areas having secondary, tertiary, and so on, constructive interference peaks. When a wavelength, or band of wavelengths, of light constructively interferes with itself, the amplitude of those wavelengths is increased, while destructively interfering wavelengths are dampened or filtered out.

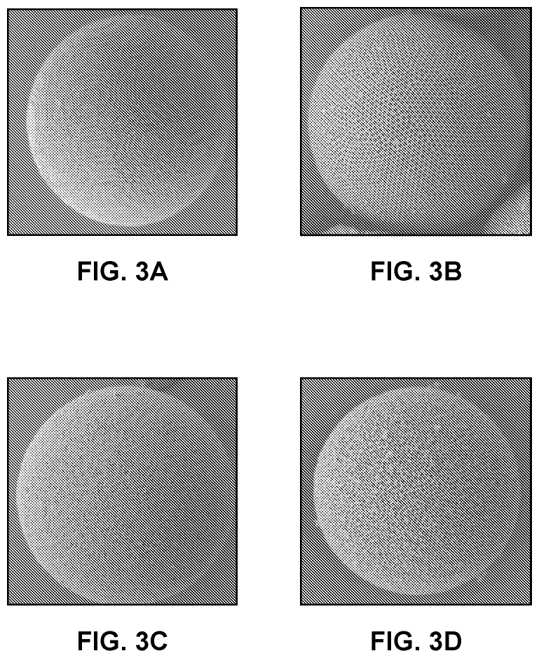

The interference pattern 210 may arise from different types of arrays of ordered or semi-ordered nanoparticles. For example, an array of nanoparticles may be an array of nanoparticles in a plane, or in a bulk material. In some embodiments an array of nanoparticles may be implemented as a microsphere made up of nanoparticles and having a particular type of structure and a bulk material may include a plurality of the microspheres containing the array of nanoparticles. FIGS. 3A through 3D are scanning microscope images of microspheres having nanoparticles with the microspheres having various structural properties. The microspheres shown have a direct, ordered structure (FIG. 3A), an inverse, ordered structure (FIG. 3B), a direct, disordered structure (FIG. 3C), and an inverse, disordered structure (FIG. 3D). The inverse structures of FIG. 3B include nanovoids confined within the microsphere. When modeling an inverse structure like a nanovoid, nanoparticles having a refractive index of 1 are used, which is the refractive index for air. Therefore, the nanoparticles shown in FIG. 3B are air which represents the nanovoids. A bulk material may include one or more of the microspheres of FIGS. 3A-3D to achieve a structural color of the bulk material. For example, a bulk material may be a dye, paint, coating, thin film, or other material having one or more of the microspheres having nanospheres as illustrated in FIGS. 3A-3D.

Each of the depicted types of structures of microspheres may have different structural color properties. For example, the ordered structures of FIGS. 3A and 3B may exhibit angle-dependent color, wherein the color observed is not the same from all angles but rather changes depending on the position of the viewer, the light source, and the sample of the ordered structures. That is, a structure that exhibits angle dependent color scatters different wavelengths of light that interfere constructive at different scattering, or reflation, angles. In contrast, the disordered structures of FIGS. 3C and 3D may exhibit angle-independent color, where the color observed is the same from all angles. That is, a same wavelength, or band of wavelengths, of light constructively interfere at all angles independent of the position of the light source, a viewer, or the sample of the disordered structures. The direct structures of FIGS. 3A and 3C may result from a three-dimensional shape of nanoparticles, or nanosurface elements (e.g., bumps, pyramids, cubes, a surface roughness, etc.) that extend outward from the microsphere, whereas the inverse structures of FIGS. 3B and 3D may result from a configuration where the surface of the microsphere is characterized by nanometer scale negative nanoparticle elements such as voids or negative spaces that extend inwards from the surface of the sphere. Polydispersity of the polymers in the nanoparticles, and surfaces of voids or negative nanoparticle elements, can give rise to disordered or semi-ordered structures, whereas monodispersity can give rise to ordered structures. In some cases, inverse structures may be more readily formulatable than direct structures, although embodiments contemplate the fabrication and use of both direct and indirect structures. It is noted, however, that not all ordered structures exhibit angle-dependent color, and not all disordered structures exhibit angle-independent color.

Table 1 below presents a listing of wavelengths of light, and the approximate nanoparticle size which can lead to constructive interference of the scattered wavelength of light, of inverse, ordered microspheres. The list of Table 1 may be useful for determining structural color parameters (e.g., void sizes) for a plurality of nanoparticles to reflect a specific wavelength of light. For example, void sizes between 200 and 240 nm may be useful for fabricating nanoparticles that scatter or reflect blue light, but further simulations and process may be required to tune the specific hue of blue, or wavelength as desired. Other features may also be useful for generating a scattered wavelength band and/or tuning the structural color of a material including void distances on a microspheres, concentrations of voids, concentrations of direct structures, sizes of direct structures, a randomized placement of voids or direct structures, periodicity of inverse or direct structures, a concentration of microspheres, or properties of a medium that contains the microspheres or nanoparticles, among others.

TABLE 1

| Type | Name | Wavelength | | Void Size | |
|---|---|---|---|---|---|
| Ultraviolet | UV | 100-400 | nm | 50-200 | nm |
| Visible Light | Violet | 400-435 | nm | 200-218 | nm |
| | Blue | 435-480 | nm | 218-240 | nm |
| | Patina | 480-490 | nm | 240-245 | nm |
| | Blue Green | 490-500 | nm | 245-250 | nm |
| | Green | 500-560 | nm | 250-280 | nm |
| | Yellow Green | 560-580 | nm | 280-290 | nm |
| | Yellow | 580-595 | nm | 290-298 | nm |
| | Orange | 595-610 | nm | 298-305 | nm |
| | Red | 610-750 | nm | 305-375 | nm |
| | Dark Red | 750-800 | nm | 375-400 | nm |
| Infrared | IR | 800-10075 | nm | 400-5038 | nm |

The scattering of light from an array of nanoparticles can be computationally difficult to model. Although Mie theory provides an accurate solution that represents the scattering of light interacting with spheres having a length scale similar to optical wavelengths of light, via Maxwell's equations, a microsphere, as described herein, is a complex object that includes a plurality of nanospheres or nanostructures. Each such nanosphere or nanostructure may scatter light in different ways, and each may have many different properties that affect the resulting Mie scattering. Consequently, some models of light scattering are not practical, or are unable, to simulate the structural color resulting from light scattering from an array of nanoparticles.

One example modeling technique for determining structural color utilizes a single-scattering approximation in which light is only scattered once by a particle of an array of particles (e.g., by a microsphere, nanosphere, by a nanoscale feature of a surface or of a microsphere, etc.). As described herein, a single scattering model simulates Mie scattering from a single nanosphere, and determines the resulting interference as the scattered light interacts with scattered light from other nearby nanospheres. The single scattering model provides information that may be indicative of reflectance peak of a particular nanosphere, microsphere, or materials having nanometer sized features. Although relatively computationally simple, the single scattering model may not accurately account for scattering in complex systems where light scatters from multiple nanospheres and may interact with other scattered light. Even so, in some circumstances the single scattering model can provide a reasonable prediction of structural color properties, and may be well-suited for simulations performed on hardware having limited processing or memory resources.

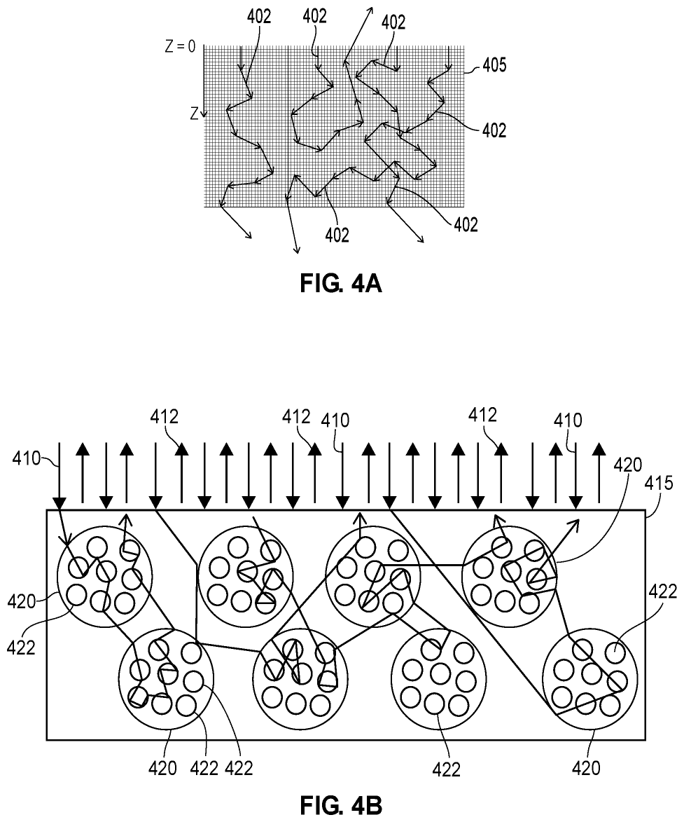

FIG. 4A is a diagram that illustrates a simulated light scattering model according to a stochastic model, such as a Monte Carlo model. The Monte Carlo model illustrated in FIG. 4A accounts for multiple scatterings of a plurality of light rays 402 as the rays 402 propagate through a medium 405. FIG. 4A illustrates that the light enters the medium 405 at the z=0 axis line, and the rays 402 propagate and scatter through the medium 405 until the rays 402 exit the medium 405 at the cutoff z axis, or at the z=0 axis. The medium 405 includes scattering elements which may include one or more microspheres, nanospheres, nanoscale surface features, voids, direct structures, indirect structures, or another scattering element or feature. The multiple scattering stochastic model depicted in FIG. 4A may be more accurate than a single scattering model while requiring only an intermediate level of computing resources. Still, the illustrated Monte Carlo model may not capture light scattering from bulk materials, or systems having numerous microspheres.

A bulk scattering model may be desired or required to determine properties of structural color of a material. An embodiment bulk model may determine a simulated reflectance from many microparticles using a two-tiered stochastic modeling approach FIG. 4B is a diagram that illustrates a bulk scattering model wherein incident 410 light is incident on, and propagates into, a bulk material 415. The bulk material 415 includes a plurality of microspheres 420, with each microsphere 420 having a plurality of nanometer scattering elements 422. Each of the nanometer scattering elements 422 may be one or more of a nanoparticle, a nanometer scale surface feature, a direct structure, an inverse structure, an ordered structure, a disordered structure, or another scattering element. The light 410 may scatter from one or more of the microspheres 420, and one or more of the nanometer scattering elements 422 multiple times before the light propagates out of the medium 415 as reflected light 412. Further, the bulk model may consider absorption of the light 410 and transmission of the light 410. Although a bulk model may require more computational resources than the single-tier scattering stochastic model or the single-scattering model, the accuracy of the predicted structural color may be improved due to the fact that the bulk model may account for properties of a bulk system.

As illustrated in FIG. 4B, and as described herein, a bulk material may be considered to include a plurality of microspheres, with each microspheres containing a plurality of nanometer scale elements, or nanoparticles. It is envisioned that the described simulation and optimization techniques may be applied to other types of bulk materials and mediums as well. For example, the disclosed methods and systems may be implemented with a bulk material containing a plurality of nanoparticles, a medium with one or more arrays of nanoparticles, a medium with one or more arrays of microparticles, a medium containing microscale and/or nanoscale scatterers, or another bulk medium capable of scattering light by microscale and/or nanoscale features.

Within a given model, various parameters may be provided for a simulation, with each parameter affecting the simulation of one or more phenomena of visible light. Each parameter may affect one or more properties of structural color of a material. A parameter may include one or more numerical values, boundary conditions, a set of values for a given variable, a number of iterations, a statistic of interest, or another type of input parameter. The phenomena of light may include, for example, a boundary condition for light scattering e.g., a spherical boundary condition, a planar boundary condition, etc.), polydispersity and mixtures of nanoparticles, light polarization, light absorption, a surface roughness, an index of refraction, a nonlinear optical coefficient, a birefringence, or an angle-dependence (e.g., a reflectance curve angle dependence, an angle dependence of a filter, an angle dependence of a grating, etc.), among others. The phenomena, and others, may be accounted for when performing a simulation using one of the above-described models.

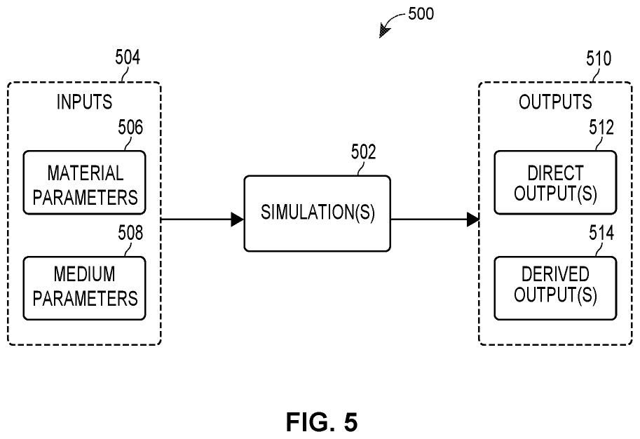

For instance, FIG. 5 illustrates an exemplary environment 500 suitable for performing simulation(s) 502 as described herein. In the environment 500, one or more inputs 504 may guide the simulation(s) 502. The inputs 504 may include various types of inputs, such as material synthesis parameters 506 that relate to the physical properties of the nanoparticles and/or one or more medium parameters 508 that relate to the properties of a grouping of nanoparticles. The one or more medium parameters 508 may include parameters pertaining to a medium that the nanoparticles are suspended in, enveloped by, or that the nanoparticles are otherwise within boundaries of the medium. Examples of material synthesis parameters 506 include, but are not limited to, optical parameters and/or structural parameters such as a surface roughness, a complex refractive index of a matrix material, an absorption of the matrix material, a dopant for the matrix material, a microparticle or nanoparticle size, a material of a nanoparticle, a volume fraction of nanoparticles, a void size, a polydispersity of nanoparticles or microparticles, or an absorber amount, a direct structure size, an inverse structure size, a nanoparticle shape/geometry, a nanoparticle shell parameter such as a shell thickness or shell material, a nanoparticle porosity, a surface feature of a nanoparticle, a lattice constant of a plurality of nanoparticles, a concentration of nanoparticles in a medium, or another parameter pertaining to a physical feature of synthesis of a material. Examples of medium parameter 508 include, but are not limited to, a type of a medium, a material of a medium, a complex refractive index of the medium, a microsphere concentration, a microsphere mixture, an absorber amount, an absorber concentration, a thickness of the array of nanoparticles, or a degree of order of the array of nanoparticles.

The inputs 504 may be provided to the simulation(s) 502, which may use the inputs 504 and a light scattering model (e.g., a bulk model, Monte Carlo model, single scattering model, a stochastic model, etc.) in order to determine approximations of various properties of visible, or non-visible, light scattered by the simulated array of nanoparticles, a bulk material, or another medium. The result of the simulation may be outputs 510. The outputs 510 may include direct output(s) 512, such as a reflectance of the array of nanoparticles at a specified wavelength or angle, or a transmittance of the array of nanoparticles at the specified wavelength. The outputs 510 may further include derived output(s) 514, which may involve multiple runs of the simulation(s) 502 in order to determine derived values of parameters. Examples of derived output(s) 514 include a reflectance curve of the array of nanoparticles, a resonance wavelength of the array of nanoparticles, an angle-dependent reflection of the array of nanoparticles, an absorption curve of the array of nanoparticles, a transmission curve of the array of nanoparticles, a range of wavelengths, a speckle amount, a scattering parameter, a target transport length, an angle independence, or a structural color of the array of nanoparticles.

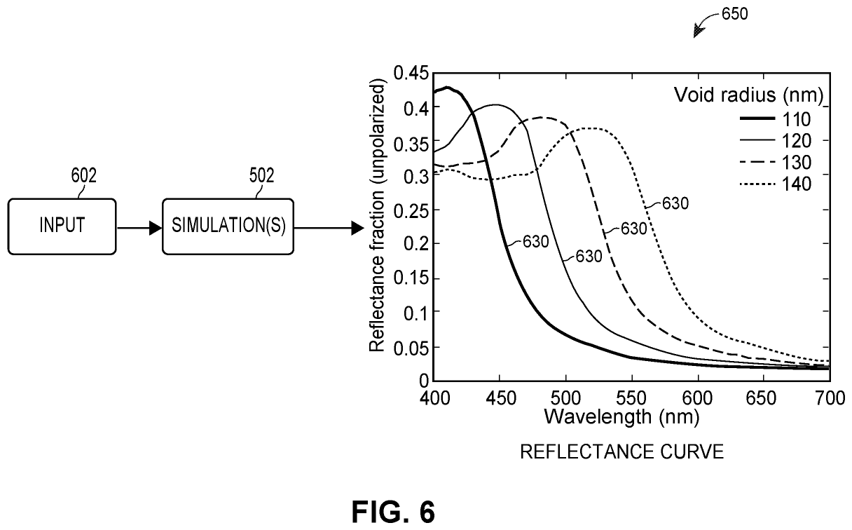

FIG. 6 illustrates an example of applying the environment 500 to generate a reflectance curve according to an exemplary embodiment. In this example, a particular set of input 602 parameters are provided and tested in multiple simulation(s) 502. For instance, a single simulation run may test the input 602 at a given wavelength, and multiple simulation runs may be used to test the input 602 over a range of wavelengths. The resulting reflectance at each tested wavelength may be used to generate a derived output in the form of a reflectance curve 630 which may be plotted and presented as shown on plot 650. Further, as shown on plot 650 of FIG. 6, the set of input parameters 602 may include a set of void sizes or void radii. The simulations 502 may be performed for a plurality of wavelengths (e.g., 400 to 700 nm) for each void size of a microparticle and a reflectance curve 630 may be determined for each void size across a range of wavelengths. While illustrated as one or more reflectance curves 730, the outputs 510 of the simulations 602 may be one or more other parameters of structural color.

The simulation procedure outlined above, for determining structural color properties of a material or an array of nanoparticles, may also be useful for determining physical and structural properties of materials to generate a target structural color. For example, in reference to FIG. 5, target outputs 510 may be identified, and an evolutionary optimization process may be performed to refine inputs 504 to achieve the target outputs 510.

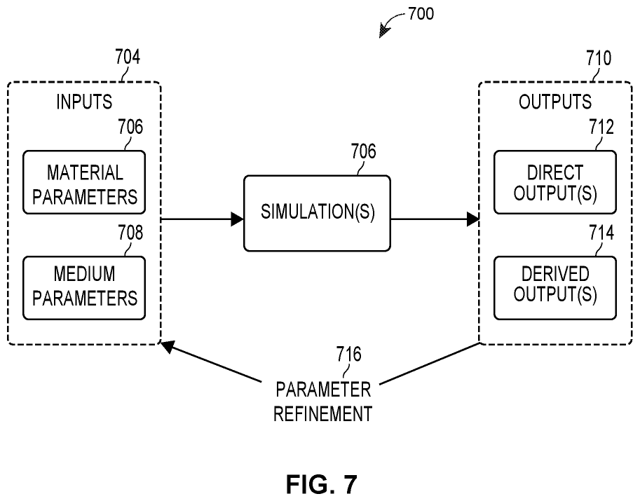

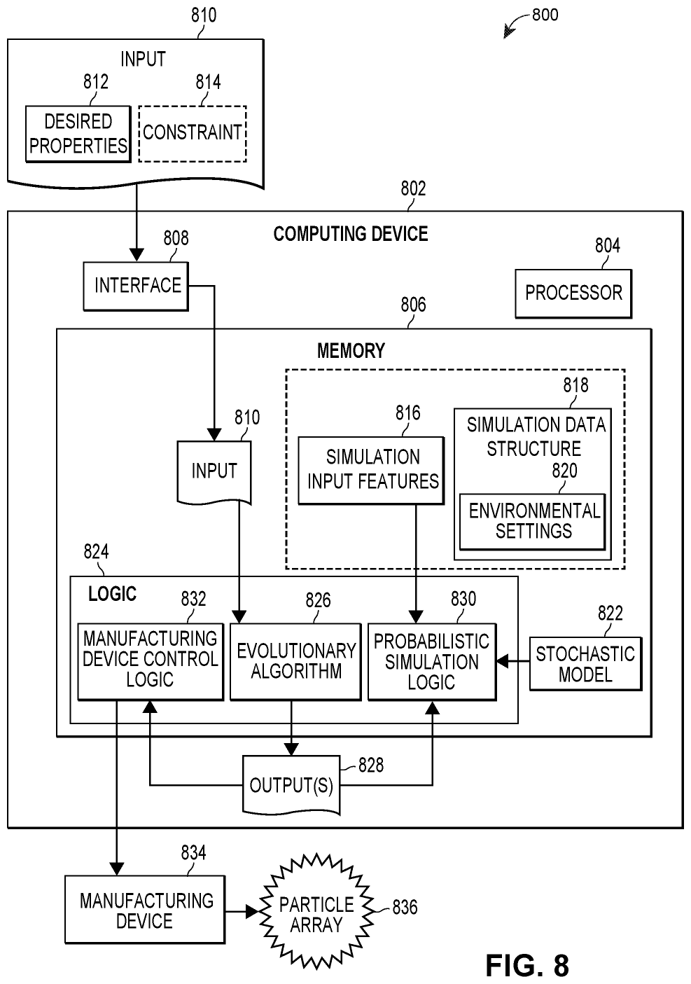

FIG. 7 is a block diagram of an environment 700 including a parameter refinement process 716 that accepts, as input, target values for the outputs 710. The parameter refinement process then selects and/or varies different inputs 704 and provides the inputs to the simulation(s) 702 in order to determine which combination of inputs 704 gives rise to the specified outputs 710. The simulations 702 may include one or more single-scattering simulation, multiple-scattering method, bulk method, another stochastic simulation or another simulation. An exemplary parameter refinement process 716 is described below in more detail with respect to FIGS. 8-10.

Turning now to a procedure for tuning parameters of an array of nanoparticles in order to achieve specified structural color properties, FIG. 8 illustrates an exemplary system 800 suitable for practicing exemplary embodiments.

The system 800 includes a computing device 802, which may be any computing device suitable for performing the processes identified below. For example, the computing device 802 may be a server, a desktop computer, a laptop, a tablet or mobile computing device, or a special-purpose computing device specifically configured to perform the identified processes. The computing device 802 may include a hardware processor 804 for executing machine executable instructions to perform 824 embodying the procedures and methods described herein. Although the illustrative computing device 802 is depicted as a single device, it is understood that the illustrated components may be distributed across multiple devices, and components illustrated as a single entity in FIG. 8 (e.g., the probabilistic simulation logic 830) may be divided between multiple computing devices.

The computing device 802 may include a memory 806, which may be a non-transitory computer-readable medium (such as RAM or ROM). Information or data may be added to the memory 806 via an interface 808 (or multiple interfaces), such as a network interface (e.g., a network interface card, Ethernet adapter, etc.), a keyboard, a mouse, a microphone, a camera, etc.

An input 810 may be provided to the interface 808. The input 810 may provide various details that drive or configure a parameter refinement process. For instance, the input 810 may specify one or more desired properties 812 of a structural color of an array of nanoparticles for at least one wavelength of light.

For example, the desired properties may include a target reflectance of the array of nanoparticles at a specified wavelength or angle, or a target transmittance of the array of nanoparticles at the specified wavelength. In some embodiments, the desired properties may include complex or derived properties, such as a target reflectance curve of the array of nanoparticles, a target resonance wavelength of the array of nanoparticles, a target angle-dependent reflection of the array of nanoparticles, a target angle independent characteristic, a scattering amount, a target absorption value or curve of the array of nanoparticles, a target transmission value or curve of the array of nanoparticles, or a target structural color of the array of nanoparticles. Exemplary embodiments can accommodate such complex/derived properties by running a probabilistic simulation multiple times (e.g., once at each wavelength, angle, etc.).

In some cases, it may be impossible or undesirable to modify one or more of the parameters of the array of nanoparticles. For example, due to constraints of a manufacturing device 834 that is used to produce a material including the array of nanoparticles. The manufacturing device 834 may not be capable of producing nanoparticles having a certain void size, may not have access to certain materials, or may not be capable of achieving a surface roughness beyond a given threshold, etc. Accordingly, the input 810 may further specify a constraint 814 that specifies values that a parameter is permitted to take, or specifies values that the parameter is not permitted to take. In some embodiments, the constraint 814 may be a hard constraint that cannot be violated (i.e., the parameter refinement process is restricted from violating the constraint 814). In others, the constraint 814 may be a soft constraint that can be violated under certain conditions (e.g., when violating the parameter yields a disproportionately large increase in achieving the desired properties, or when the desired properties cannot be achieved without violating the constraint 814).

The memory 806 may further store simulation input features 816 used to define or guide the simulation. The simulation input features 816 may include a parameter for the stochastic model, a Fresnel reflection approach, a complex refractive index dispersion equation, a number of Monte Carlo trajectories, a number of Monte Carlo events, an effective medium approximation strategy, or a selection for a type of the array of nanoparticles.

The memory 806 may further store a simulation data structure 818 that defines an environmental setting 820 for the probabilistic simulation. The environmental setting may be, for example, a boundary condition, a polarization, or a medium for the array of nanoparticles. The simulation data structure 818 may be applied when conducting the probabilistic simulation on the array of nanoparticles to determine one or more simulated properties of the structural color of the array of nanoparticles.

In order to determine how visible (or non-visible) light interacts with an array of nanoparticles, a stochastic model 822 of the scattering of the light may be applied, as described above. In some embodiments, the stochastic model may be a Monte Carlo model.

In order to select or refine the parameters, instructions executable on the processor 804 may be stored in the memory 806 in the form of logic 824. For instance, the logic 824 may include an evolutionary algorithm 826, probabilistic simulation logic 830, and/or manufacturing device control logic 832.

The evolutionary algorithm 826 may be an algorithm for refining the parameters of the array of nanoparticles using mechanisms corresponding to biological evolutionary principles (e.g., reproduction, mutation, recombination, and selection). The evolutionary algorithm 826 may be a population-based optimization algorithm. The evolutionary algorithm 826 may be a non-gradient-based evolutionary algorithm that does not rely on a gradient of the objective function when refining the parameters. One example of a non-gradient-based evolutionary algorithm is a differential evolution algorithm that iteratively improves the parameters with respect to a cost function. In embodiments, the evolutionary algorithm may include a maximum likelihood optimization, a differential evolution algorithm, a Bayesian optimization, a genetic algorithm, a covariance matrix adaptation evolution strategy, a particle swarm optimization, and simulated/or annealing.

The evolutionary algorithm 826 may generate one or more output(s) 828 representing values for the parameters of the array of nanoparticles. The output(s) 828 may be evaluated by probabilistic simulation logic 830, which runs a probabilistic simulation of an array of nanoparticles having properties corresponding to the output(s) 828 using the stochastic model 822. The results of the probabilistic simulation may be fed back to the evolutionary algorithm 826 (e.g., to be analyzed in connection with a cost function) to determine whether the evolutionary algorithm 826 is improving the parameters with respect to the desired properties 812.

When a stopping condition is met, the evolutionary algorithm 826 may generate a final set of output(s) 828. The stopping condition may include a value associated with a cost function, a statistic of interest, a difference between one or more inputs 810 and one or more of the outputs 828, a number of iterations of the evolutionary algorithm, or another stopping condition. Optionally, the output(s) 828 may be provided to the manufacturing device control logic 832, which may be configured to control a manufacturing device 834 to produce material including an array of nanoparticles 836 having parameters corresponding to the output(s) 828. For example, the manufacturing device 834 may be configured to produce a bulk material including one or more of a dye, paint, or coating including an array of nanoparticles having structural and physical features corresponding to the output(s) 828.

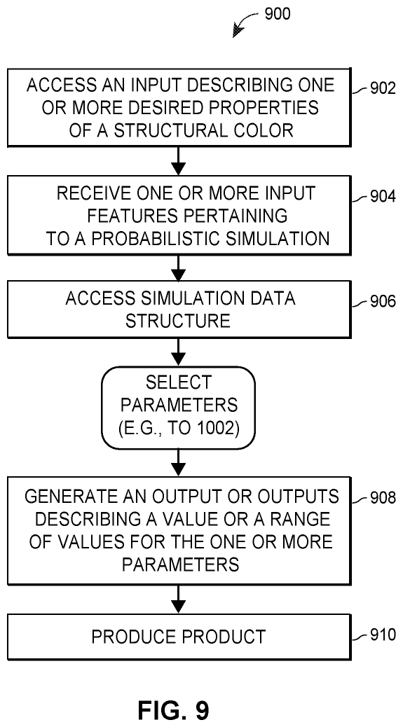

FIG. 9 illustrates a method 900 for selecting parameters of the array of nanoparticles in accordance with an embodiment.

In block 902, the method 900 may access an input describing one or more desired properties of a structural color of an array of nanoparticles for at least one wavelength of light. The array of nanoparticles may be, for example, an arrangement of nanoparticles (such as a sheet or plane), a part of a microsphere, or a bulk material comprising a plurality of microspheres. The bulk material may be a dye, ink, paint, coating, or another material medium that contains the plurality of microspheres, and/or a plurality of nanospheres.

In block 904, the method 900 may include receiving one or more input features pertaining to a probabilistic simulation of a behavior of the light applied to the array of nanoparticles. The probabilistic simulation may apply a stochastic model of the array of nanoparticles.

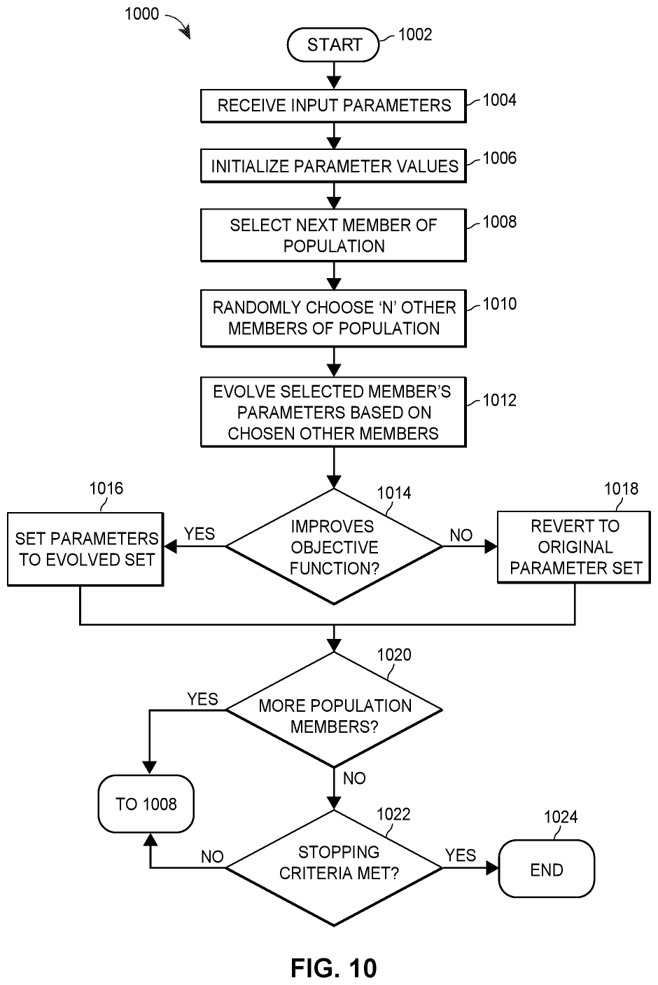

In block 906, the method 900 includes accessing a simulation data structure defining an environmental setting for the probabilistic simulation. The environmental setting may be, for example, a boundary condition, a polarization, or a medium for the array of nanoparticles. The simulation data structure may be used to conduct the probabilistic simulation on the array of nanoparticles to determine one or more simulated properties of the structural color of the array of nanoparticles, as described in more detail in connection with FIG. 10. For instance, the probabilistic simulation may be run using the one or more input features to quantitatively predict observable color structure features of the array of nanoparticles. By applying an optimization technique such as a differential evolution algorithm, the probabilistic simulation may be implemented over multiple iterations and one or more parameters of the array of nanoparticles may be adjusted for each iteration to approximate the desired properties of the structural color.

At block 908, based on applying the stochastic model, the method 900 generates an output indicative of a single value, multiple values, or a range of values for the one or more parameters of the array of nanoparticles to approximate the desired properties of the structural color for the at least one wavelength of light.

At block 910, the method 900 may include controlling machinery to produce a product including an array of nanoparticles having parameters that have been determined using the above-described process according to the method 900. For example, the machinery may produce a dye, a paint, or a coating comprising the array of nanoparticles.

Structural color arising from nanoparticles can result in especially bright, saturated colors that appear very similar to colors produced by more conventional dyes, paints, and coatings that rely on absorption to produce a color. Accordingly, conventional dyes, paints, and coatings can readily be replaced with similar products incorporating structural color, which has a number of advantages over absorption-based color. Structural color resists photobleaching and may be less photoreactive. Moreover, different structural colors can be made from the same base materials by changing the properties of the nanoparticles (e.g., the size of the voids and/or the size of the particles). As a result, a user can first select the component materials that will be used to make a particular color, based on non-color properties (e.g., toxicity, reactivity, etc.), and can then produce different colors from those materials by altering the properties of the nanoparticles.

FIG. 10 illustrates a method 1000 for tuning the parameters of an array of nanoparticles according to an exemplary embodiment. The method 1000 applies a differential evolution algorithm, which is one example of a non-gradient-based evolutionary algorithm. In evolutionary algorithms generally, and differential evolution algorithms specifically, one or more parameters are optimized based on an objective function (e.g., a minimization function, maximization function, difference value, hit target, etc.) that is improved in a series of iterations. At each iteration, decision variables are updated (i.e., evolved) through various rules and heuristics, depending on the algorithm.

In differential evolution, each parameter set is modified based on a set of rules (e.g., a percent change, a random change, a perturbation, a weighted difference modification, a modification by one or more factors, etc.), and depending on the improvement of the objective function with these changes, the modified parameter set is either rejected or accepted. The set of rules may be akin to biologic evolution. For instance, a particular parameter may be randomly combined with values in other parameter sets (i.e., crossover). This may be performed at every iteration, leading to a new population of parameter sets for subsequent evaluation. The iterations halt after a stop point (e.g., a user-defined stopping criterion, predetermined number of iterations, etc.). Differential evolution is useful for optimizing parameters for systems with continuous parameter sets, a large number of variables, and objective functions.

Differential evolution may be implemented to optimize structural color parameters. The ultimate goal in structural color design is often to achieve a targeted structural color (e.g., reflectance spectrum) by varying the various physical and optical parameters of the structural color material. The combination of differential evolution and the above-described probabilistic simulation may be implemented to determine parameters of nanoparticles and materials to achieve a desired structural color. In an example, probabilistic simulation logic may be embedded into a differential evolution framework. First, a user may enter a target reflectance spectrum (e.g., reflectance values at given wavelengths). Other inputs may include: (1) one or more parameters of interest to vary to achieve the target; (2) for said parameters, the range to be explored; and (3) differential evolution parameters. Using the inputs, the differential evolution algorithm may initialize various parameter sets. For each parameter set, the stochastic model may be executed and may use the parameter values to predict reflectance at specified wavelengths. The system may compare the determined output reflectance spectrum to the target reflectance spectrum. The system may determine a difference between the output reflectance spectrum and the target reflectance spectrum as an objective function value for a specific parameter set. The system may execute the stochastic model for each parameter set in the population to determine output reflectance spectrums for each parameter set. The differential evolution algorithm may then update a family of structural color parameter sets via the rules outlined above (e.g., perturbation, percent change, weighted change values, etc.), and a next iteration of the evolution optimization may be performed. Through subsequent iterations, the objective function may be minimized to reduce the difference between the output and target reflectance spectra. At a stopping point (e.g., number of iterations, threshold of a cost or objective function, etc.) one or more output spectra, and the optimized physical and optical parameters and associated values to achieve the output spectra, may be recorded. The differential evolution optimization procedure described above is discussed in more detail below with reference to the method 1000.

Referring simultaneously to both FIGS. 8 and 10, the method 1000 begins at block 1002. Processing may proceed to block 1004, where the system receives a set of input parameters, which may include a list of parameters to be optimized, maximum and/or minimum values for the parameters, etc. The parameters may correspond to parameters of the array of nanoparticles that can be changed in order to affect the structural observable color of the array of nanoparticles (e.g., structural parameter values, structural features, optical parameter values, etc.). The parameters of the array of nanoparticles may include, but are not limited to, material synthesis parameters that relate to the physical properties of the nanoparticles and/or medium parameters that relate to the properties of a grouping of nanoparticles. Examples of material synthesis parameters include, but are not limited to, optical parameters and/or structural parameters including a surface roughness, a complex refractive index of a matrix material, an absorption of the matrix material, a dopant for the matrix material, a microparticle or nanoparticle size, a volume fraction of nanoparticles, a void size, a polydispersity of particles, an absorber amount, a nanoparticle shell parameter (e.g., a shell thickness and/or a material of the shell), a nanoparticle shape/geometry, a direct structure size, an indirect structure size, nanoparticle porosity, a surface feature of a nanoparticle, a lattice constant of a plurality of nanoparticles, a concentration of nanoparticles in a medium, or another parameter pertaining to a physical, optical, or electrical feature of a material. Examples of medium parameters include, but are not limited to, a type of a medium, a complex refractive index of the medium, a microsphere concentration, a microsphere mixture, an absorber amount, an absorber concentration, properties of the absorber, a thickness of the array of nanoparticles, a thickness of a bulk material made up of the array of nanoparticles, a thickness of a microsphere made up of the array of nanoparticles, a thickness of a bulk material made up of microspheres, or a degree of order of the array of nanoparticles. The properties of the absorber may include, for example, optical parameters such as an absorption coefficient or complex refractive index dispersion behavior of the absorber.

At block 1006, the parameter values are initialized. A population of M members is established, where M is an integer value, which may be predetermined or may be dynamically determined based on, for example, currently available processing resources. Each member of the population may be associated with a set of the parameters where each of the parameters in the set, for a given member, may have a different value as compared to a corresponding parameter for a different member of the population. The parameter values may be initialized (e.g., randomly, taking into account the acceptable minimum and maximum values, assigned or input by a user, etc.) for each member of the population.

In some embodiments, an input, such as the input 810 of FIG. 8, may specify a starting parameter space in which parameters may be further refined. A starting parameter space may be particularly useful, for example, if a user has identified a set of parameters giving rise to a particular color, but wishes to further refine one or more properties of the color (e.g., making the color more saturated, change the hue). Accordingly, when initializing the parameters at block 1006, the method 1000 may include selecting parameter values that fall within the identified starting parameter space. If a starting parameter space is identified for some, but not all, parameters, then those parameters for which a starting parameter space has been identified may be initialized within that space, and the remaining parameters may be initialized randomly or by another means. The parameters may be permitted to leave the starting parameter space or may be constrained to remain within the starting parameter space, depending on the application.

At block 1008, the system begins to evolve the parameter values for a given member of the population. The next member of the population, or the first member, during the first iteration of the algorithm, is selected for processing.

At block 1010, the system randomly chooses n other members of the population whose parameters will be used to evolve the selected member's parameter values. In one embodiment outlined in the pseudocode below, the value of n may be three.

At block 1012, the system evolves the selected member's parameter set based on corresponding parameter values of the other chosen members. Evolving the parameters may involve combining the chosen members' parameter values based on a function, with the possibility of including a random element, through random perturbation, or by another evolution rule or process. An example of evolving the selected member's parameter is provided in the pseudocode below. Block 1012 may be performed for each parameter in the parameter set.

In block 1012, the system may optionally enforce the constraint 814, or multiple constraints, when adjusting the parameter values. In addition to “box constraints” (i.e., min/max limits on each parameter), differential evolution is also capable of handling other various types of constraints, such as constraints on combinations of parameters. For example, a constraint may enforce certain size range when a certain material is used, but a different size range when another material. In some embodiments, constraints may be “soft constraints” that can be violated only if the resulting improvement in the objective function exceeds a predetermined threshold. Soft constraints allow a constraint to be violated, but requires that the benefit accrued from violating the constraint be worth the associated cost.

At decision block 1014, the system determines whether the evolved parameter set improves an objective function as compared to the previous version of the parameter value assigned to the member. The objective function may be a function that accepts a parameter set and an objective (e.g., the desired properties of visible light scattered by the array of nanoparticles, as defined in the input 810) and applies a probabilistic simulation using a stochastic model based on the parameter set. The probabilistic simulation may account for the particular form of the array of nanoparticles (e.g., nanoelements of a single microsphere, of bulk microspheres). The objective function may rank, score, or otherwise assign a quantitative value representing how closely an array of nanoparticles having the parameters defined by the parameter set matches the desired properties of the visible light.

Among other possibilities, the objective function may be applied to reduce undesired light scattering by the array of nanoparticles, the undesired light scattering represented as a cost function that is minimized by evolutionary optimization, maximize reflectance at a specified wavelength, reduce reflectance of undesired light at unspecified wavelengths, or maximize light scattering over visible wavelengths. The objective function may determine these quantitative values via the probabilistic simulation.

If the determination at decision block 1014 is “YES,” then processing may proceed to block 1016 and the system may set the parameter values for the current member to the evolved values determined in block 1012. If the determination at decision block 1014 is “NO,” then processing may proceed to block 1018, and the parameter values may revert to their original, unevolved values. Processing may then proceed to decision block 1020.

At decision block 1020, the system may determine if additional population members remain to be evaluated. If so, processing reverts to block 1008 and the next member of the population is selected. If not, processing proceeds to decision block 1022, and the system determines whether a predefined stopping criteria has been met. The predefined stopping criteria, also referred to as a stop condition, may include, for instance, whether a predetermined number of iterations have been performed, whether a predetermined amount of time has elapsed, whether the objective function rises or falls to a predetermined threshold indicating that an acceptable solution has been found, or whether the improvement between evolutions of the parameter values is increasing or decreasing at a predefined rate, indicating that the parameter values are not improving over the previous m iterations, a determination of stagnation of the objective function over a predefined number of iterations, among other possibilities. If the stopping criteria has not yet been met, then processing may revert to block 1008 and the system may revert back to the first member of the population. Otherwise, processing may proceed to block 1024 and terminate.

A particular implementation of the method 1000 may be represented by the following pseudocode:

```
set input parameters to algorithm
  M = number of members of the population (number of parameter set candidates)
  Q = number of iterations
  CR = crossover probability (ranging from 0 to 1, inclusive)
  F = differential weight (ranging from 0 to 2, inclusive)
Initialize the parameter values of each member of the population
(with p number of parameters (dimension of p))
For i = 1 : Q
  For j = 1: M
    Choose at random, 3 other (distinct) members of the population (m [symbol] , m [symbol] , m [symbol] )
    Choose, at random, an index (L) from 1:p
    Calculate member, `s potentially new parameter set, n
      For k = 1:p
        Choose a uniform random number r [symbol]  (r [symbol]  ~U(0,1))
        If r [symbol]  < CR or k = L
          set n [symbol]  (kth parameter of potentially
          new jth member) accoring to : n [symbol]  =
          m [symbol]  + F [symbol] (m [symbol]  − m [symbol] )
        Else
          Do nothing
    Calculate objective function (f) for member n [symbol] . If
    f(n [symbol] ) ≤ f(m [symbol] ), replace the m [symbol]  with the new n [symbol]
```

[symbol]

indicates data missing or illegible when filed

There are numerous benefits to the described evolutionary optimization based determination of nanoparticle properties. First, in general, evolutionary algorithms are not gradient based, and therefore do not have to rely on the gradient of the objective function, which can often be costly in computational resources to update and improve. Furthermore, they do not assume anything about the objective function, and can handle challenging systems, including those that are multi-modal, stochastic, and non-differentiable. Also, the described evolutionary optimization based approach attempts to find the global, as opposed to the local, optimum. Specifically, differential evolution also has the advantage that it can determine multiple solutions instead of a single solution, which is useful when the practitioner cannot use the “best” solution for numerous reasons-including feasibility issues, cost constraints, a fabrication constraint, etc. —which may not have been included in the optimization algorithm.

In some embodiments, the parameter tuning method 1000 may be employed to effect a color that is the result of a mixture of different types of arrays of ordered or semi-ordered nanoparticles. For example, a first microsphere exhibiting a first set of structural color properties may be mixed with a second microsphere exhibiting a second set of structural color properties. The mixture may be associated with scattered light representing a mixture of the first and second structural color properties, resulting in a third structural color different than either of the constituent first or second structural colors. The properties of the third structural color may be measured and used to guide parameter selection using the method 1000. In this way, the method 1000 may select parameters for a single array of nanoparticles that exhibit the third structural color without relying on the combination of the first and second microspheres.

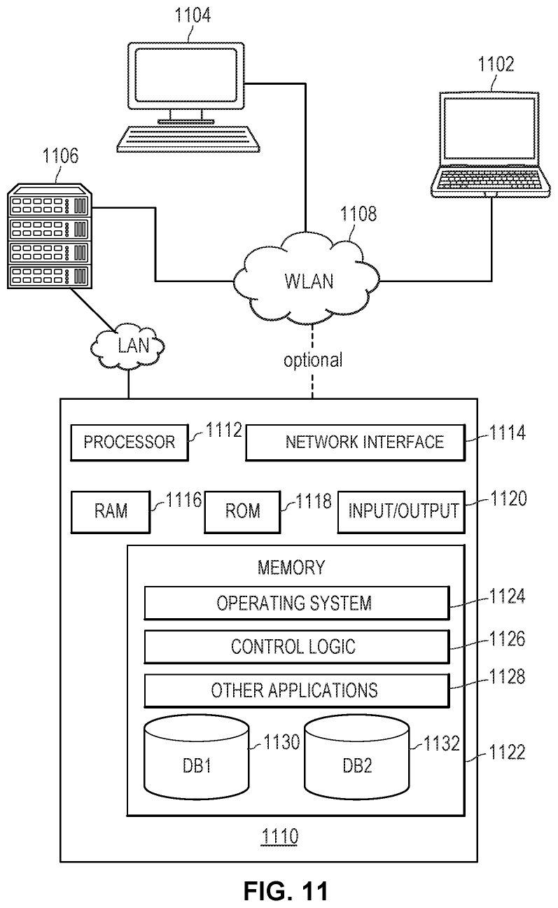

FIG. 11 illustrates one example of a system architecture and data processing device that may be used to implement one or more illustrative aspects described herein in a standalone and/or networked environment. Various network nodes, such as the data server 1110, web server 1106, computer 1104, and laptop 1102 may be interconnected via a wide area network 1108 (WAN), such as the internet. Other networks may also or alternatively be used, including private intranets, corporate networks, LANs, metropolitan area networks (MANs) wireless networks, personal networks (PANs), and the like. Network 1108 is for illustration purposes and may be replaced with fewer or additional computer networks. A local area network (LAN) may have one or more of any known LAN topology and may use one or more of a variety of different protocols, such as ethernet. Devices data server 1110, web server 1106, computer 1104, laptop 1102 and other devices (not shown) may be connected to one or more of the networks via twisted pair wires, coaxial cable, fiber optics, radio waves or other communication media.

Computer software, hardware, and networks may be utilized in a variety of different system environments, including standalone, networked, remote-access (aka, remote desktop), virtualized, and/or cloud-based environments, among others.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data—attributable to a single entity—which resides across all physical networks.

The components may include data server 1110, web server 1106, and client computer 1104, laptop 1102. Data server 1110 provides overall access, control and administration of databases and control software for performing one or more illustrative aspects described herein. Data server 1110 may be connected to web server 1106 through which users interact with and obtain data as requested. Alternatively, data server 1110 may act as a web server itself and be directly connected to the internet.

Data server 1110 may be connected to web server 1106 through the network 1108 (e.g., the internet), via direct or indirect connection, or via some other network. Users may interact with the data server 1110 using remote computer 1104, laptop 1102, e.g., using a web browser to connect to the data server 1110 via one or more externally exposed web sites hosted by web server 1106. Client computer 1104, laptop 1102 may be used in concert with data server 1110 to access data stored therein, or may be used for other purposes. For example, from client computer 1104, a user may access web server 1106 using an internet browser, as is known in the art, or by executing a software application that communicates with web server 1106 and/or data server 1110 over a computer network (such as the internet).

Servers and applications may be combined on the same physical machines, and retain separate virtual or logical addresses, or may reside on separate physical machines. FIG. 11 illustrates just one example of a network architecture that may be used, and those of skill in the art will appreciate that the specific network architecture and data processing devices used may vary, and are secondary to the functionality that they provide, as further described herein. For example, services provided by web server 1106 and data server 1110 may be combined on a single server.

Each component data server 1110, web server 1106, computer 1104, laptop 1102 may be any type of known computer, server, or data processing device. Data server 1110, e.g., may include a processor 1112 controlling overall operation of the data server 1110. Data server 1110 may further include RAM 1116, ROM 1118, network interface 1114, input/output interfaces 1120 (e.g., keyboard, mouse, display, printer, etc.), and memory 1122. Input/output interfaces 1120 may include a variety of interface units and drives for reading, writing, displaying, and/or printing data or files. Memory 1122 may further store operating system software 1124 for controlling overall operation of the data server 1110, control logic 1126 for instructing data server 1110 to perform aspects described herein, and other application software 1128 providing secondary, support, and/or other functionality which may or may not be used in conjunction with aspects described herein. The control logic 1126 may also be referred to herein as the data server software control logic 1126. Functionality of the data server software may refer to operations or decisions made automatically based on rules coded into the control logic 1126, made manually by a user providing input into the system, and/or a combination of automatic processing based on user input (e.g., queries, data updates, etc.).

Memory 1122 may also store data used in performance of one or more aspects described herein, including a first database 1132 and a second database 1130. In some embodiments, the first database may include the second database (e.g., as a separate table, report, etc.). That is, the information can be stored in a single database, or separated into different logical, virtual, or physical databases, depending on system design. Web server 1106, computer 1104, laptop 1102 may have similar or different architecture as described with respect to data server 1110. Those of skill in the art will appreciate that the functionality of data server 1110 (or web server 1106, computer 1104, laptop 1102) as described herein may be spread across multiple data processing devices, for example, to distribute processing load across multiple computers, to segregate transactions based on geographic location, user access level, quality of service (QoS), etc.

Graphical User Interface (GUI)

Some embodiments relate to a GUI for use in determining optical properties of a nanoparticle or nanoparticle array, and/or for use in determining one or more structural properties of an array of nanoparticles that result in desired optical properties for a structural color of the array.

Example System

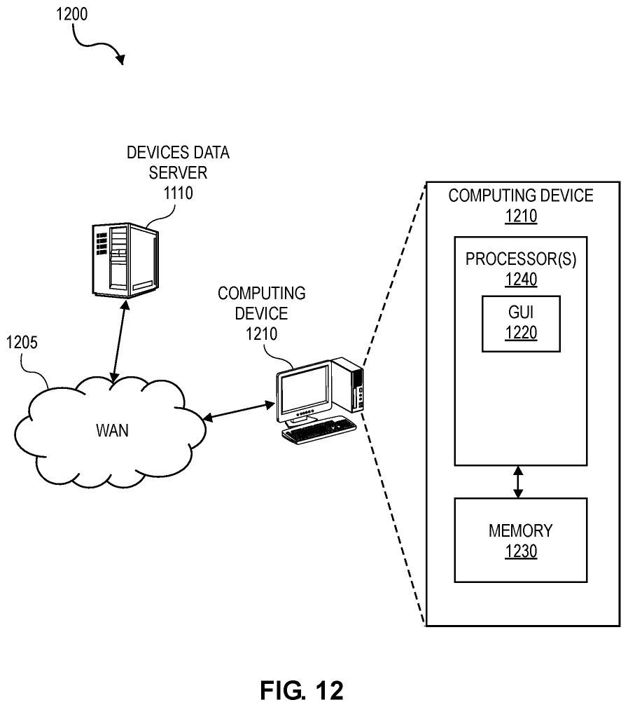

FIG. 12 illustrates one example of a system architecture 1200 that may be used to implement one or more illustrative aspects described herein relating to the GUI. As illustrated in the example of FIG. 12, the data server 1110, and computing device 1210 may be interconnected via a wide area network 1205 (WAN), such as the internet. Other networks may also or alternatively be used, including private intranets, corporate networks, LANs, metropolitan area networks (MANs) wireless networks, personal networks (PANs), and the like. Network 1205 is for illustration purposes and may be replaced with fewer or additional computer networks. A local area network (LAN) may have one or more of any known LAN topology and may use one or more of a variety of different protocols, such as ethernet. Devices data server 1110, computing device 1210 (e.g., a mobile phone, smartphone, laptop, computer, etc.) and other devices (not shown) may be connected to one or more of the networks via twisted pair wires, coaxial cable, fiber optics, radio waves or other communication media.

The example system 1200 includes a computing device 1210, which may be any computing device suitable for performing the processes identified below. For example, the computing device 1210 may be a mobile computing device, a desktop computer, a laptop, a tablet or server, or a special-purpose computing device specifically configured to perform the identified processes. The computing device 1210 may include a hardware processor 1240 for running the GUI 1220 for performing the techniques described herein. Although the illustrative computing device 1210 is depicted as a single device, it is understood that the illustrated components may be distributed across multiple devices, and components illustrated as a single entity in FIG. 12 may be divided between multiple computing devices.

The computing device 1210 may include a memory 1230, which may be a non-transitory computer-readable medium (such as RAM or ROM). Information or data may be added to the memory 1230 via an interface (or multiple interfaces), such as a network interface (e.g., a network interface card, Ethernet adapter, etc.), a keyboard, a mouse, a microphone, a camera, etc.

The processor may run a GUI 1220 in accordance with the techniques described herein. In this regard, and as will be described below, the GUI 1220 may be run in a forward configuration, or an inverse configuration. In some embodiments, in the forward configuration, an optical effect is determined from a physical parameter or optical parameter; and, in the inverse configuration, a physical parameter or optical parameter is determined from an optical effect. Put another way, in one example, in the forward configuration, the GUI 1220 accepts an input parameter that is a physical property or optical property of a nanoparticle material (e.g., nanoparticle size, shape, etc.), and outputs an optical effect of the nanoparticle material (e.g., a color, a graph of a reflectance vs. wavelength, etc.); whereas, in the inverse configuration, the GUI 1220 accepts the optical effect as the input, and outputs a physical property or optical property of the nanoparticle material.

Forward Configuration Embodiments

In the forward configuration, in some embodiments, a user may enter input properties (e.g., inputs indicative of a physical property of a plurality of nanoparticles, such as structural properties of the nanoparticles) into the GUI 1220; and the GUI 1220 may in turn display an output optical parameter (e.g., a wavelength, range of wavelengths, reflection curve, transmission curve, absorption curve, a speckle amount, a scattering parameter, etc.) based on the input properties.

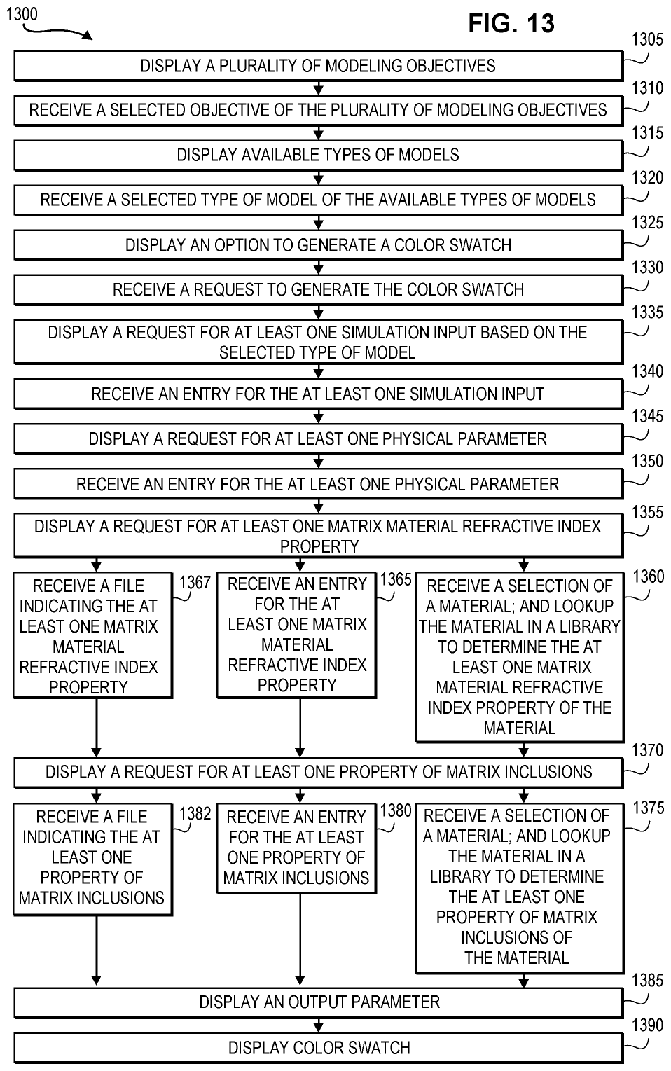

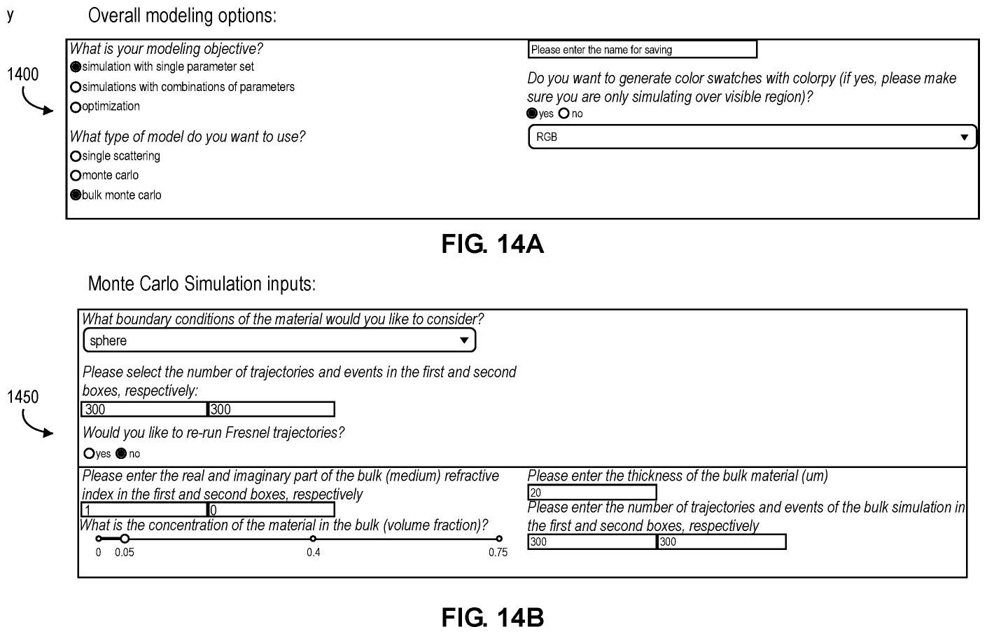

FIG. 13 illustrates an example method 1300 of a forward configuration. With reference thereto, at block 1305, the method 1300 begins by displaying a plurality of modeling objectives. In some embodiments, the modeling objectives are displayed as part of an overall modeling options screen, an example of which is shown by screen 1400 of FIG. 14A. In some implementations, the example screen 1400 is the first screen that a user sees when the GUI 1220 is launched. As illustrated in the example of FIG. 14A, in some embodiments, the modeling objectives include: a simulation with a single parameter set; simulations with combinations of parameters; and/or optimization. With a single parameter set; the user enters one combination of the parameters of the system, with the output being the optical effect arising from that parameter set. With a combination of parameters, the user enters numerous values of one or more parameters (e.g., void size of 200 nm, void size of 220 nm, nanoparticle size of 162 nm, etc.), with the output being numerous optical effects, each arising from one combination of physical parameters. With optimization, the user performs mathematical optimization through the inverse configuration to obtain parameters which yield a target optical effect.

At block 1310, the GUI 1220 receives a user selection of one of the modeling objectives. In one example of this, the user may select a modeling objective from a bullet point list (e.g., as in the example of screen 1400).

At block 1315, the GUI 1220 displays available types of models. In some embodiments, the available types of models are displayed as part of the overall modeling options screen (e.g., as in screen 1400 of FIG. 14A). In some implementations, the types of models include: single scattering; Monte Carlo; and/or bulk Monte Carlo.

At block 1320, the GUI 1220 receives a selected type of model from the available types of models. For instance, the user may select a type of model by clicking on the type of model, as in the example screen 1400.

At block 1325, the GUI 1220 displays an option to generate a color swatch. In some embodiments, a color swatch comprises an illustration of a single color. In other embodiments, the color swatch comprises a palette of colors.

At block 1330, the GUI 1220 receives a request to generate the color swatch. For instance, the user may click on “yes,” as in the example of screen 1400. In addition, the user may specify that the color swatch is to be an RGB color swatch, as in the example screen 1400. The user can select other color models to generate the color swatch, including iRGB and CIE.

At block 1335, the GUI 1220 displays a request for at least one simulation input based on the selected type of model. In some embodiments, the at least one simulation input includes: a real and imaginary part of a bulk refractive index; a concentration of microspheres in a bulk; and/or a thickness of the bulk (e.g., where the bulk is a nanoparticle material).

In some implementations, the simulation inputs may be based on the selected type of model. For instance, when the selected type of model is a Monte Carlo or bulk Monte Carlo, in addition to simulation inputs for other types of models, the simulation inputs may further include a boundary condition (e.g., a spherical boundary condition or a planar boundary condition), the number of trajectories and/or events, and/or a prompt asking if Fresnel trajectories should be re-run. The boundary conditions describe the geometry of the material which include the nanoparticles, and affects the scattering of the light.

In another example, when the selected type of model is bulk Monte Carlo, in some implementations, the simulation inputs may further include a number of bulk events, a number of bulk trajectories, a concentration of microspheres in the formulation, a thickness of the coating, and/or real and/or imaginary refractive index of the bulk medium.

At block 1340, the GUI 1220 receives an entry for the at least one simulation input. For example, the user may make an entry into a screen, such as the example screen 1450. The user may make the entry in any suitable manner. For instance, the GUI 1220 may allow the user to make the entry by using drop down arrow(s), typing the entry into a box, and/or using a slider bar.

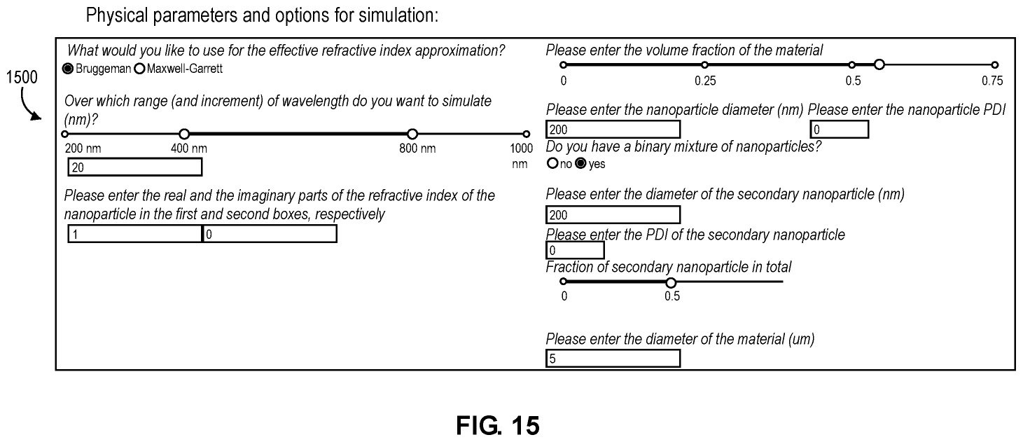

At block 1345, the GUI 1220 displays a request for at least one physical parameter, as in example screen 1500 of FIG. 15. In some embodiments, the at least one physical parameter comprises a wavelength range; a real and imaginary part of a refractive index of a nanoparticle; a microsphere volume fraction; a primary nanoparticle diameter; a secondary nanoparticle diameter; a fraction of secondary nanoparticles of a nanoparticle total; or a microparticle diameter. In this regard, it should be understood that simulations may be run on a composition that includes both a primary nanoparticle material and a secondary nanoparticle material.

In some embodiments, the physical parameter selection includes a selection between if the effective refractive index approximation should be made by a Bruggeman approximation, or a Maxwell-Garnett approximation.

In some embodiments, the at least one physical parameter comprises a polydispersity index (PDI) of the primary nanoparticle, and/or a PDI of the secondary nanoparticle.

At block 1350, the GUI 1220 receives an entry for the at least one physical parameter. For example, the user may make an entry into a screen, such as the example screen 1500. The user may make the entry in any suitable manner. For instance, the GUI 1220 may allow the user to make the entry by using drop down arrow(s), typing the entry into a box, and/or using a slider bar.

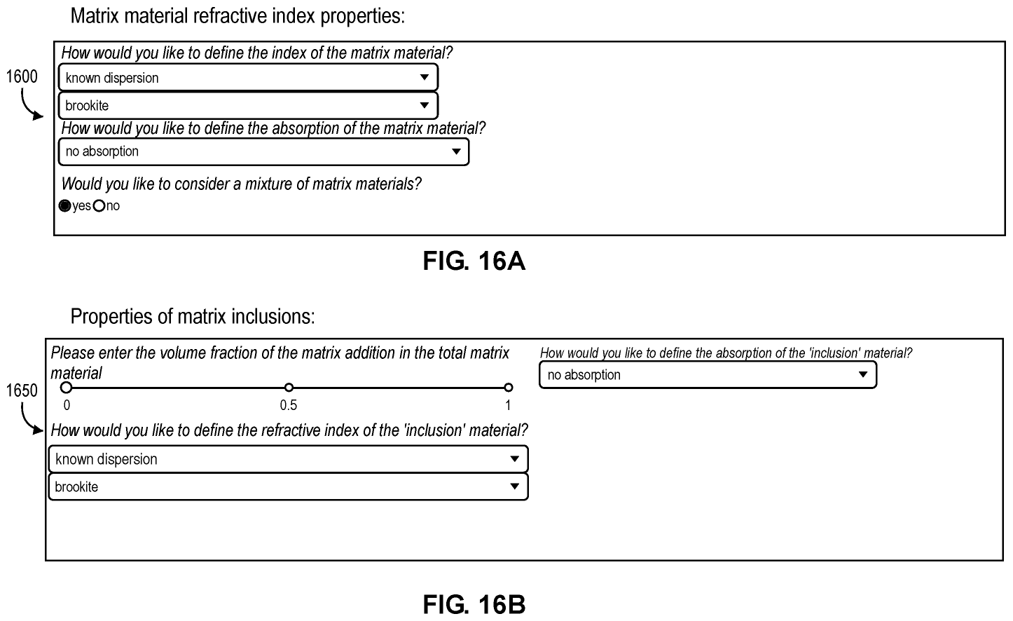

At block 1355, the GUI 1220 displays a request for at least one matrix material refractive index property. To respond to this request, in some embodiments, the user has three options. As a first option, the user may specify a material (e.g., by selecting from a drop down menu, by typing the name of the material, etc.); and the GUI 1220 will access a library (e.g., a database) of materials to find the at least one refractive index property (block 1360). For instance, the user may, from a dropdown menu, select Brookite, as in the example screen 1600 of FIG. 16A.

As a second option, the user may simply manually enter the at least one matrix material refractive index property (block 1365). The user may make the entry in any suitable manner. For instance, the GUI 1220 may allow the user to make the entry by using drop down arrow(s), typing the entry into a box, and/or using a slider bar.

Aspects of this are also illustrated in the example screen 1600.

As a third option, the user may use or upload a file (e.g., a.csv file) which specifies the wavelength dependence of the refractive index (block 1367). In one example of this, the user may have generated such a file in previous experiments.

In some embodiments, the at least one matrix material refractive index property defines an index of a matrix material. In some embodiments, the at least one matrix material refractive index property defines an absorption of a matrix material.

At block 1370, the GUI 1220 displays a request for at least one property of matrix inclusions. An example of this is shown in screen 1650 of FIG. 16B. In some embodiments, the at least one property of matrix inclusions comprises a volume fraction of a matrix addition in a total matrix material. In some embodiments, the at least one property of matrix inclusions comprises a refractive index of an inclusion material. In some embodiments, the at least one property of matrix inclusions comprises an absorption of an inclusion material.

To respond to this request, in some embodiments, the user has three options. As a first option, the user may specify a material (e.g., by selecting from a drop down menu, by typing the name of the material, etc.); and the GUI 1220 will access a library (e.g., a database) of materials to find the at least one property of matrix inclusions (block 1375). For instance, the user may, from a dropdown menu, select Brookite, as in the example screen 1650 of FIG. 16B.

As a second option, the user may simply manually enter the at least one property of matrix inclusions (block 1380). The user may make the entry in any suitable manner. For instance, the GUI 1220 may allow the user to make the entry by using drop down arrow(s), typing the entry into a box, and/or using a slider bar. Aspects of this are also illustrated in the example screen 1650.

As a third option, the user may use or upload a file (e.g., a.csv file) which specifies the wavelength dependence of the refractive index (block 1382). In one example of this, the user may have generated such a file in previous experiments.

At block 1385, the GUI 1220 displays an output parameter. The output parameter may be calculated by any suitable technique, such as the techniques described herein.

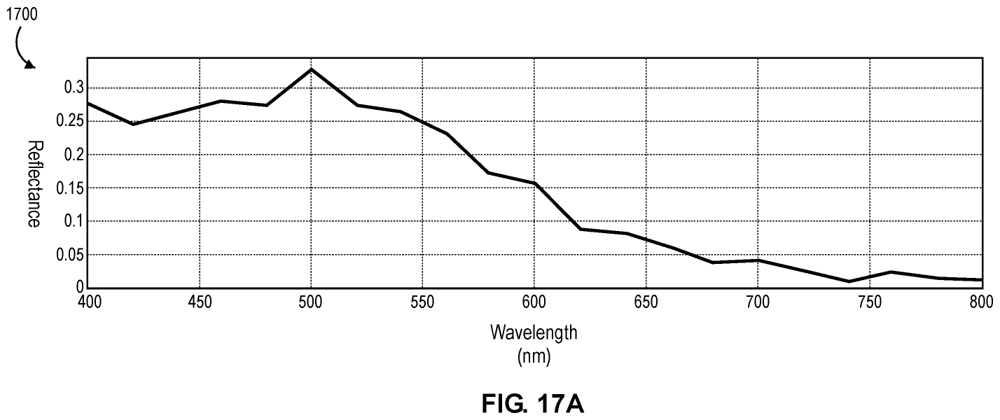

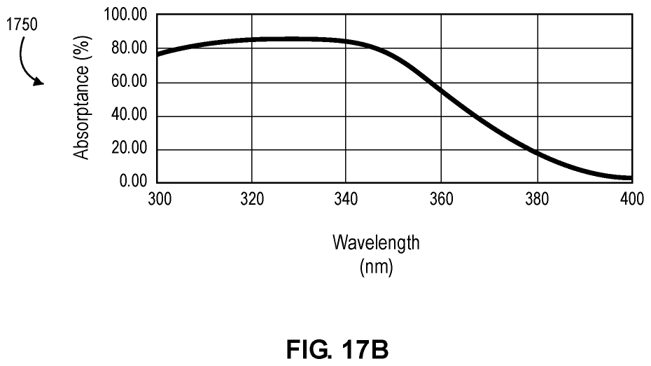

In some embodiments, the output parameter comprises a graph of a reflectance vs. wavelength, such as in example screen 1700 of FIG. 17A. In some embodiments, the output parameter may be a numerical value(s) indicating the reflectance.

In some embodiments, the output parameter comprises a graph of an absorption vs. wavelength, such as in example screen 1750 of FIG. 17B. In some embodiments, the output parameter may be a numerical value(s) indicating the absorption. Advantageously, the output parameter being an absorption facilitates the determination of a color (e.g., absorption, or lack thereof, of a particular wavelength of light will change what color(s) are reflected back to the viewer). Further advantageously, the output parameter being an absorption facilitates the determination of if ultra-violet (UV) light will be absorbed (e.g., the graph of absorption vs. wavelength shows if/how well UV light will be absorbed).

In some embodiments, the output parameter is simply a color or range of colors. This may be displayed by displaying the color(s) itself, or by displaying text indicating the color (e.g., displaying the word “red”).

In some implementations, the output parameter may be generated based on any or all of: (i) the selected objective, (ii) the selected type of model, (iii) the entry for the at least one simulation input, (iv) the entry of the at least one physical parameter, (v) the received entry for the at least one matrix material refractive index property, and/or (vi) the received entry for the at least one property of matrix inclusions.

At block 1390, the GUI 1220 displays the color swatch (if it was requested by the user). The color swatch may be displayed in any suitable way (see above discussion with respect to blocks 1325, 1330).

Inverse Configuration Embodiments

In the inverse configuration, in some embodiments, a user may enter optical property objectives, decision variables, and optimization parameters; and, in response, the GUI 1220 may output an optimized material property, or optimized reflectance curve.

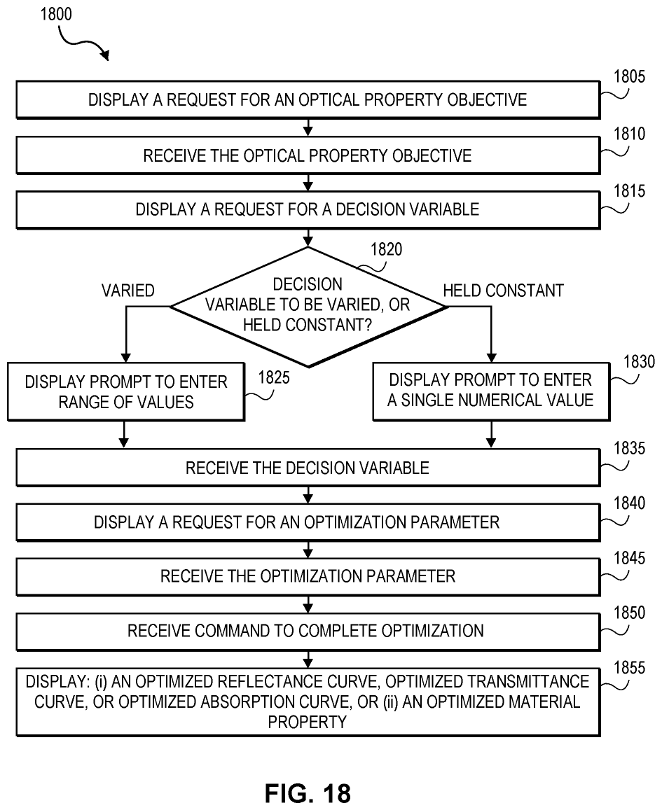

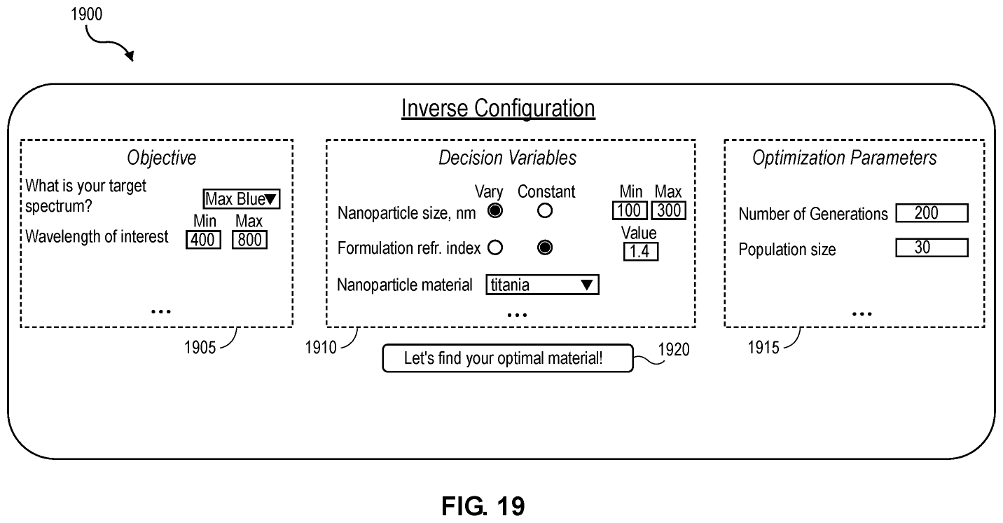

FIG. 18 illustrates an example method 1800 of an inverse configuration. With reference thereto, at block 1805, the method 1800 begins by displaying a request for an optical property. In some embodiments, this is done as illustrated in optical property objective box 1905 of example screen 1900 of FIG. 19. It should be understood that although the example of FIG. 19 illustrates two optical property objectives (e.g., “Max Blue,” and a specified wavelength range of interest), any number of optical property objectives may be used. For instance, when a user presses the “ . . . ” in the optical property objective box 1905, the optical property objective box 1905 may expand to display more optical property objectives.

In some embodiments, the displayed request comprises a question, such as the question “What would you like to do,” as in the example of screen 1900. The displayed request may further allow a user to select from a dropdown menu, also as in the example of screen 1900. In some embodiments, the drop down menu presents options of: matching a target spectrum; maximizing or minimizing a reflectance ratio; or maximizing or minimizing an area under a reflectance curve ratio.

At block 1810, the GUI 1220 receives the optical property objective. In some embodiments, upon receipt of the optical property objective, the GUI 1220 may prompt the user for additional information. For example, if the user indicates that the optical property objective is matching a target spectrum, the GUI 1220 may prompt the user to enter a target reflectance curve, transmission curve, and/or absorption curve. In some embodiments, the user may enter the target reflectance cure in any suitable format (e.g., a.csv format, etc.). It should be understood that the entered target reflectance curve may then be added to the received optical property objective.

In another example, if the received optical property objective comprises maximizing a reflectance ratio or maximizing an area under a reflectance curve ratio, the GUI 1220 may prompt the user to input a wavelength range. In some embodiments, the GUI 1220 allows the user to input the wavelength range in “Min” and “Max” boxes, such as in the example of screen 1900. Additionally or alternatively, buttons may allow a user to increment wavelength range values. In some embodiments, the wavelength range may be entered by inputting a reflectance curve, such as in a.csv format. In some embodiments, the reflectance curve is adjustable by the user.

At block 1815, the GUI 1220 displays a request for a decision variable. In some embodiments, this comprises presenting options to the user to hold a decision variable constant, or vary the decision variable. The user may select between these options using any suitable technique. For instance, in decision variables box 1910 of the example screen 1900, the user is allowed to make this selection via radio buttons.

In some embodiments, the GUI 1220 may further prompt the user for information based on if the decision variable is to be varied or constant (e.g., block 1820). For example, if the decision variable is to be varied, the GUI 1220 may prompt the user to enter a range of values (e.g., a void size range, nanoparticle size range, etc.) (e.g., block 1825). The user may specify this range using any suitable technique. For instance, the user may enter the values into text boxes, such as the “Min” and “Max” text boxes in the example decision variable box 1910.

In another example, in some embodiments, if the decision variable is to be held constant (e.g., block 1830), the user may enter a numerical value into a text box, such as in the “Value” box in the example decision variable box 1910.

Furthermore, in some embodiments, the decision variable may be a continuous decision variable. In this regard, in some implementations, the continuous decision variable comprises at least one of: nanoparticle size, nanoparticle absorption, nanoparticle refractive index, volume fraction of the microsphere, microsphere size, refractive index of the bulk medium, concentration of the microspheres, or thickness of the bulk film.

In some embodiments, the decision variable is a discrete decision variable. For instance, the decision variable may be a matrix material. In this regard, in some implementations, the user selects the matrix material (e.g., by using a dropdown menu, by typing the matrix material name into a text box, etc.); and, the GUI 1220 then accesses a library (e.g., a database) to determine one or more properties of the matrix material, such as a matrix material refractive index. Furthermore, in some embodiments, if the user decides to enter the matrix material as a constant decision variable, the user would select a single matrix material from the library; and, if the user decides to enter the matrix material as a varied decision variable, the user may select multiple matrix materials from the library.

It should further be understood that although the example of FIG. 19 illustrates two decision variables (e.g., nanoparticle size, and formulation refractive index), any number of decision variables may be used. For instance, when a user presses the “ . . . ” in the decision variables box 1910, the box 1910 may expand to display more decision variables. In addition, in some embodiments, the decision variable is a void size of a nanoparticle material. Here, in some implementations, it should be understood that the text in decision variables box 1910 may indicate “Void size” instead of or in addition to “Nanoparticle size.”

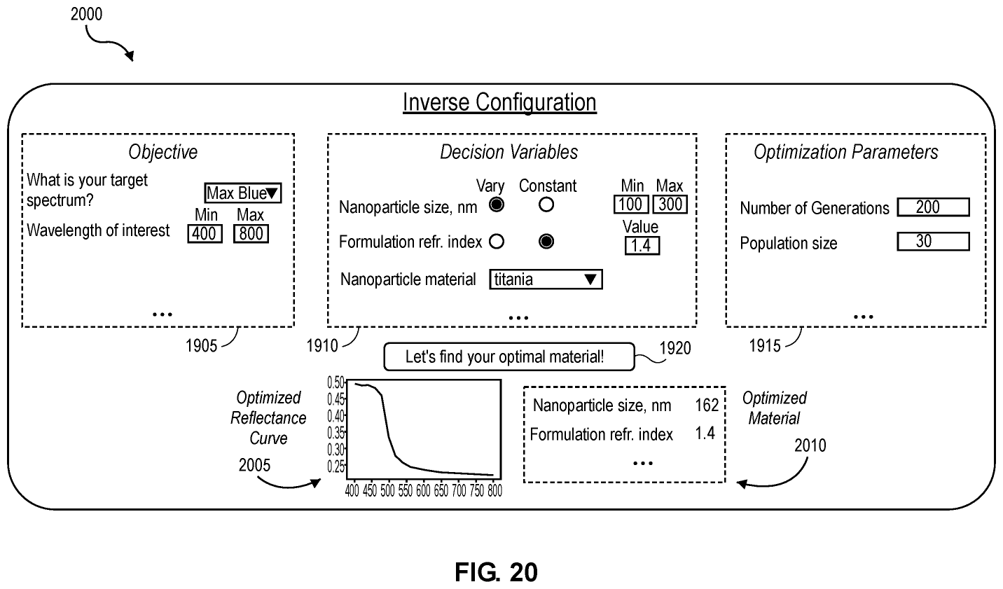

Furthermore, a user may enter a name of a nanoparticle material into the decision variables box 1910 (e.g., by typing the name of the nanoparticle material, or selecting it from a list, such as from a list expanded via a dropdown arrow). In this regard, the nanoparticle material may be a decision variable, and thus the displayed output parameter may be displayed further based on the nanoparticle material. In the examples of FIGS. 19 and 20, the nanoparticle material has been specified to be titania; however, it should be understood that any type of nanoparticle material may be specified. In some embodiments, the decision variables box 1910 first displays a prompt to the user asking if the user would like to enter: (i) only the nanoparticle size and refractive index, or (ii) enter the nanoparticle size and the nanoparticle material. The decision variables box 1910 may then display the appropriate entry options depending on the user's answer.

At block 1835, the GUI 1220 receives the decision variable from the user. The decision variable may be received, for example, as described above, including information of if the decision variable is to be varied or if the variable is to be held constant, information of a wavelength range, etc. In some implementations, if the (decision) variable is to be held constant, the system can refer to this as simply the “variable,” rather than the “decision variable.”

At block 1840, the GUI 1220 displays a request for an optimization parameter. For example, the GUI 1220 may display an optimization parameters box, such as the example optimization parameters box 1915.

In some embodiments, the optimization parameters comprise evolutionary parameters. For example, the optimization parameters may comprise: a number of generations; a population size; a crossover probability; or a weighting factor.

It should further be understood that although the example of FIG. 19 illustrates two optimization parameters (e.g., number of generations, and population size), any number of optimization parameters may be used. For instance, when a user presses the “ . . . ” in the optimization parameters box 1915, the box 1915 may expand to display more optimization parameters.

At block 1845, the GUI 1220 receives the optimization parameter input by the user.

At block 1850, the GUI receives a command to complete the optimization.

This may occur by any suitable technique. For example, the user may press button 1920 of screen 1900.

At block 1855, the GUI 1220 displays: (i) an optimized reflectance curve, optimized transmittance curve, or optimized absorption curve or (ii) an optimized material property. In some implementations, the (i) optimized reflectance curve, or (ii) an optimized material property was generated based on the optical property objective, the decision variable, and the optimization parameter (e.g., using the techniques described herein).

In this regard, FIG. 20 shows an example screen 2000, including optimized reflectance curve 2005, and optimized material property 2010. In other words, the GUI 1220 displays screen 2000 after a user has pressed button 1920 in screen 1900. In some embodiments, the optimized reflectance curve comprises a graph of a reflectance vs. wavelength. Furthermore, although the example of FIG. 20 illustrates an example reflectance curve, it should be understood that the screen 2000 may additionally or alternatively display transmittance curve(s) and/or absorption curve(s). In some embodiments, the displayed optimized material indicates a nanoparticle size; void size of a nanoparticle material; or a formulation refractive index of the nanoparticle material. In some embodiments, the displayed optimized material indicates nanoparticle size, nanoparticle absorption, nanoparticle refractive index, volume fraction of the microsphere, microsphere size, refractive index of the bulk medium, concentration of the microspheres, and/or thickness of the bulk film.

Furthermore, in some embodiments, combinations of parameters may be simulated. For example, the screen 1900 may allow a user to enter any number of combinations of optical property objective(s) (e.g., into optical property objective box 1905), decision variable(s) (e.g., into decision variables box 1910), and/or optimization parameter(s) (e.g., into optimization parameters box 1915). The screen 1900 may keep accepting inputs of the combinations of parameters until the user indicates that she is finished entering inputs. Subsequently, the screen 2000 may display any number of reflectance curve(s), transmittance curve(s), absorption curve(s), and/or optimized material properties based on the input combinations. Advantageously, this may be used in a sensitivity analysis of the inputs. For example, displaying multiple curves and or optimized property objectives allows for a user to determine which parameters have a greater effect on the curves and/or optimized property objectives. In this way, the user may “screen” inputs (e.g., determine which inputs to focus on).

Forward Configuration Aspects

Aspect 1. A computer-implemented method, comprising:
displaying, by a processor, a plurality of modeling objectives;
receiving, at the processor, a selected objective of the plurality of modeling objectives;
displaying, by the processor, available types of models;
receiving, at the processor, a selected type of model of the available types of models;
displaying, by the processor, a request for at least one simulation input based on the selected type of model;
receiving, at the processor, an entry for the at least one simulation input; displaying, by the processor, a request for at least one physical parameter; receiving, at the processor, an entry for the at least one physical parameter; and displaying, by the processor, an output parameter, wherein the output parameter was generated based on: (i) the selected objective, (ii) the selected type of model, (iii) the entry for the at least one simulation input, and (iv) the entry of the at least one physical parameter.

Aspect 2. The computer-implemented method of aspect 1, wherein the output parameter comprises a graph of a reflectance vs. wavelength.

Aspect 3. The computer-implemented method of aspects 1 or 2, further comprising:
displaying, by the processor, a request for at least one matrix material refractive index property; and receiving, at the processor, an entry for the at least one matrix material refractive index property;
wherein the displaying of the output parameter is further based on the received entry for the at least one matrix material refractive index property.

Aspect 4. The computer-implemented method of aspect 3, wherein the entry for the at least one matrix material refractive index property defines an index of a matrix material.

Aspect 5. The computer-implemented method of aspect 3 or 4, wherein the entry for the at least one matrix material refractive index property defines an absorption of a matrix material.

Aspect 6. The computer-implemented method of any one of aspects 1-5, further comprising:
receiving, at the processor, a selection of a material; and accessing, by the processor, a library of materials to determine at least one matrix material refractive index property of the material; and wherein the displaying of the output parameter is further based on the at least one matrix material refractive index property of the material determined from the library of materials.

Aspect 7. The computer-implemented method of any one of aspects 1-6, further comprising:
displaying, by the processor, a request for at least one property of matrix inclusions;
receiving, at the processor, an entry for the at least one property of matrix inclusions;
wherein the displaying of the output parameter is further based on the received entry for the at least one property of matrix inclusions.

Aspect 8. The computer-implemented method of any of aspects 1-7, wherein the at least one property of matrix inclusions comprises a volume fraction of a matrix addition in a total matrix material.

Aspect 9. The computer-implemented method of aspect 7, wherein the at least one property of matrix inclusions comprises a refractive index of an inclusion material.

Aspect 10. The computer-implemented method of aspect 7 or 8, wherein the at least one property of matrix inclusions comprises an absorption of an inclusion material.

Aspect 11. The computer-implemented method of any one of aspects 1-10, wherein the plurality of modeling objectives include:
a simulation with a single parameter set;
simulations with combinations of parameters; or optimization.

Aspect 12. The computer-implemented method of any one of aspects 1-11, wherein the types of models include:
single scattering; Monte Carlo; or bulk Monte Carlo.

Aspect 13. The computer-implemented method of any one of aspects 1-12, further comprising:
receiving, at the processor, a request to generate a color swatch;
in response to receiving the request to generate the color swatch, generating a color swatch based upon the output parameter.

Aspect 14. The computer-implemented method of any one of aspects 1-13, wherein the selected type of model is a Bulk Monte Carlo model, and the at least one simulation input includes:
a real and imaginary part of a bulk refractive index;
a concentration of microspheres in a bulk;
a thickness of the bulk;
number of bulk trajectories;
a number of bulk events; or a shell thickness and material.

Aspect 15. The computer-implemented method of any one of aspects 1-14, wherein the selected type of model is a Monte Carlo model, and the at least one simulation input includes:
a boundary condition comprising a spherical boundary condition or a planar boundary condition;
a number of trajectories;
a number of events; or a shell thickness and material.

Aspect 16. The computer-implemented method of any one of aspects 1-15, wherein the at least one physical parameter comprises:
a wavelength range;
a real and imaginary part of a refractive index of a nanoparticle;
a microsphere volume fraction;
a primary nanoparticle diameter;
a secondary nanoparticle diameter;
a fraction of secondary nanoparticles of a nanoparticle total; or a microparticle diameter.

Aspect 17. The computer-implemented method of any one of aspects 1-16, wherein the at least one simulation input includes a shell thickness and material.

Aspect 18. A computer system comprising one or more processors configured to:
display a plurality of modeling objectives;
receive a selected objective of the plurality of modeling objectives;
display available types of models;
receive a selected type of model of the available types of models;
display a request for at least one simulation input based on the selected type of model; receive an entry for the at least one simulation input;
display a request for at least one physical parameter;
receive an entry for the at least one physical parameter; and display an output parameter, wherein the output parameter was generated based on: (i) the selected objective, (ii) the selected type of model, (iii) the entry for the at least one simulation input, and (iv) the entry of the at least one physical parameter.

Inverse Configuration Aspects

Aspect 19. A computer-implemented method, comprising:
displaying, by a processor, a request for an optical property objective;
receiving, at the processor, the optical property objective;
displaying, by the processor, a request for a decision variable;
receiving, at the processor, the decision variable;
displaying, by the processor, a request for an optimization parameter;
receiving, at the processor, the optimization parameter; and displaying, by the processor: (i) an optimized reflectance curve, optimized transmittance curve, or optimized absorption curve, or (ii) an optimized material property, wherein the displayed (i) optimized reflectance curve, or (ii) optimized material property was generated based on the optical property objective, the decision variable, and the optimization parameter.

Aspect 20. The computer-implemented method of aspect 19, wherein the optical property objective comprises:
matching a target spectrum;
maximizing or minimizing a reflectance ratio;
maximizing or minimizing an area under a reflectance curve ratio;
matching a color; or matching a diffuse transmission.

Aspect 21. The computer-implemented method of aspect 19 or 20, wherein the request for the optical property objective comprises:
a prompt regarding matching a target spectrum; and a prompt to input a target reflectance curve.

Aspect 22. The computer-implemented method of any one of aspects 19-21, wherein:
the receiving of the optical property objective comprises receiving a response of maximizing a reflectance ratio; and
the method further comprises:
in response to the optical property objective comprising maximizing a reflectance ratio, prompting a user to input a wavelength range;
wherein the optical property objective further comprises a wavelength range input by the user.

Aspect 23. The computer-implemented method of any one of aspects 19-22, wherein:
the receiving of the optical property objective comprises receiving a response of maximizing an area under a reflectance curve ratio; and
the method further comprises:
in response to the optical property objective comprising maximizing an area under a reflectance curve ratio, prompting a user to input a wavelength range; wherein the optical property objective further comprises a wavelength range input by the user.

Aspect 24. The computer-implemented method of any one of aspects 19-23, wherein the displaying the request for the decision variable comprises presenting an options to vary and hold constant decision variables.

Aspect 25. The computer-implemented method of any one of aspects 19-24, wherein:
the receiving of the decision variable comprises receiving an indication that a first decision variable should be varied; and
the method further comprises:
in response to receipt of the indication that the first decision variable should be varied, displaying a prompt to enter a decision variable value range.

Aspect 26. The computer-implemented method of any one of aspects 19-25, wherein:
the receiving of the decision variable comprises receiving an indication that a first decision variable should be held constant; and
the method further comprises:
in response to receipt of the indication that the first decision variable should be held constant, displaying a prompt to enter a single numerical value.

Aspect 27. The computer-implemented method of any one of aspects 19-26, wherein the decision variable comprises at least one of:
nanoparticle size, nanoparticle absorption, nanoparticle refractive index, volume fraction of the microsphere, microsphere size, refractive index of the bulk medium, concentration of the microspheres, or thickness of the bulk film.

Aspect 28. The computer-implemented method of any one of aspects 19-27, wherein the decision variable comprises a matrix material, and the method further comprises:
accessing, by the processor, a library of materials to determine at least one matrix material refractive index property of the matrix material.

Aspect 29. The computer-implemented method of any one of aspects 19-27, wherein the decision variable comprises a matrix material specified by a continuous variable.

Aspect 30. The computer-implemented method of any one of aspects 19-29, wherein the optimization parameter comprises a differential evolution parameter comprising:
a number of generations;
a population size;
a crossover probability; or a weighting factor.

Aspect 31. The computer-implemented method of any one of aspects 19-30, wherein the optimized reflectance curve is displayed, and comprises a graph of a reflectance vs. wavelength.

Aspect 32. The computer-implemented method of any one of aspects 19-31, wherein the optimized material property is displayed, and comprises at least one of: a nanoparticle size;
a void size of a nanoparticle material; or a formulation refractive index of the nanoparticle material.

Aspect 33. The computer-implemented method of any one of aspects 19-32, wherein the optimized material property is displayed, and comprises at least one of: nanoparticle absorption, nanoparticle refractive index, volume fraction of the microsphere, microsphere size, refractive index of the bulk medium, concentration of the microspheres, or thickness of the bulk film.

Aspect 34. The computer-implemented method of any one of aspects 19-33, wherein the decision variable comprises a name of a nanoparticle material.

Aspect 35. A computer system comprising one or more processors configured to:
display a request for an optical property objective;
receive the optical property objective;
display a request for a decision variable;
receive the decision variable;
display a request for an optimization parameter;
receive the optimization parameter; and display: (i) an optimized reflectance curve, optimized transmittance curve, or optimized absorption curve, or (ii) an optimized material property, wherein the displayed (i) optimized reflectance curve, or (ii) optimized material property was generated based on the optical property objective, the decision variable, and the optimization parameter.

Other Matters

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time.

For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations.

Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

What is claimed:

1. A computer-implemented method, comprising:
displaying, by a processor, a plurality of modeling objectives;
receiving, at the processor, a selected objective of the plurality of modeling objectives;
displaying, by the processor, available types of models;
receiving, at the processor, a selected type of model of the available types of models;
displaying, by the processor, a request for at least one simulation input based on the selected type of model;
receiving, at the processor, an entry for the at least one simulation input;
displaying, by the processor, a request for at least one physical parameter;
receiving, at the processor, an entry for the at least one physical parameter; and
displaying, by the processor, an output parameter, wherein the output parameter was generated based on: (i) the selected objective, (ii) the selected type of model, (iii) the entry for the at least one simulation input, and (iv) the entry of the at least one physical parameter.

2. The computer-implemented method of claim 1, wherein the output parameter comprises a graph of a reflectance vs. wavelength.

3. The computer-implemented method of claim 1, further comprising:
displaying, by the processor, a request for at least one matrix material refractive index property; and
receiving, at the processor, an entry for the at least one matrix material refractive index property;
wherein the displaying of the output parameter is further based on the received entry for the at least one matrix material refractive index property.

4. The computer-implemented method of claim 3, wherein the entry for the at least one matrix material refractive index property defines an index of a matrix material.

5. The computer-implemented method of claim 3, wherein the entry for the at least one matrix material refractive index property defines an absorption of a matrix material.

6. The computer-implemented method of claim 1, further comprising:
receiving, at the processor, a selection of a material; and
accessing, by the processor, a library of materials to determine at least one matrix material refractive index property of the material; and
wherein the displaying of the output parameter is further based on the at least one matrix material refractive index property of the material determined from the library of materials.

7. The computer-implemented method of claim 1, further comprising:
displaying, by the processor, a request for at least one property of matrix inclusions;
receiving, at the processor, an entry for the at least one property of matrix inclusions;
wherein the displaying of the output parameter is further based on the received entry for the at least one property of matrix inclusions.

8. The computer-implemented method of claim 7, wherein the at least one property of matrix inclusions comprises a volume fraction of a matrix addition in a total matrix material.

9. The computer-implemented method of claim 7, wherein the at least one property of matrix inclusions comprises a refractive index of an inclusion material.

10. The computer-implemented method of claim 7, wherein the at least one property of matrix inclusions comprises an absorption of an inclusion material.

11. The computer-implemented method of claim 1, wherein the plurality of modeling objectives include:
a simulation with a single parameter set;
simulations with combinations of parameters; or
optimization.

12. The computer-implemented method of claim 1, wherein the types of models include:
single scattering;
Monte Carlo; or
bulk Monte Carlo.

13. The computer-implemented method of claim 1, further comprising:
receiving, at the processor, a request to generate a color swatch;
in response to receiving the request to generate the color swatch, generating a color swatch based upon the output parameter.

14. The computer-implemented method of claim 1, wherein the selected type of model is a Bulk Monte Carlo model, and the at least one simulation input includes:
a real and imaginary part of a bulk refractive index;
a concentration of microspheres in a bulk;
a thickness of the bulk;
number of bulk trajectories;
a number of bulk events; or
a shell thickness and material.

15. The computer-implemented method of claim 1, wherein the selected type of model is a Monte Carlo model, and the at least one simulation input includes:
a boundary condition comprising a spherical boundary condition or a planar boundary condition;
a number of trajectories;
a number of events; or
a shell thickness and material.

16. The computer-implemented method of claim 1, wherein the at least one physical parameter comprises:
a wavelength range;
a real and imaginary part of a refractive index of a nanoparticle;
a microsphere volume fraction;
a primary nanoparticle diameter;
a secondary nanoparticle diameter;
a fraction of secondary nanoparticles of a nanoparticle total; or
a microparticle diameter.

17. The computer-implemented method of claim 1, wherein the at least one simulation input includes a shell thickness and material.

18. A computer system comprising one or more processors configured to:
display a plurality of modeling objectives;
receive a selected objective of the plurality of modeling objectives;
display available types of models;
receive a selected type of model of the available types of models;
display a request for at least one simulation input based on the selected type of model;
receive an entry for the at least one simulation input;
display a request for at least one physical parameter;
receive an entry for the at least one physical parameter; and
display an output parameter, wherein the output parameter was generated based on: (i) the selected objective, (ii) the selected type of model, (iii) the entry for the at least one simulation input, and (iv) the entry of the at least one physical parameter.

19. A computer-implemented method, comprising:
displaying, by a processor, a request for an optical property objective;
receiving, at the processor, the optical property objective;
displaying, by the processor, a request for a decision variable;
receiving, at the processor, the decision variable;
displaying, by the processor, a request for an optimization parameter;
receiving, at the processor, the optimization parameter; and
displaying, by the processor: (i) an optimized reflectance curve, optimized transmittance curve, or optimized absorption curve, or (ii) an optimized material property, wherein the displayed (i) optimized reflectance curve, or (ii) optimized material property was generated based on the optical property objective, the decision variable, and the optimization parameter.

20. The computer-implemented method of claim 19, wherein the optical property objective comprises:
matching a target spectrum;
maximizing or minimizing a reflectance ratio;
maximizing or minimizing an area under a reflectance curve ratio;
matching a color; or
matching a diffuse transmission.

21. The computer-implemented method of claim 19, wherein the request for the optical property objective comprises:
a prompt regarding matching a target spectrum; and
a prompt to input a target reflectance curve.

22. The computer-implemented method of claim 19, wherein:
the receiving of the optical property objective comprises receiving a response of maximizing a reflectance ratio; and
the method further comprises:
in response to the optical property objective comprising maximizing a reflectance ratio, prompting a user to input a wavelength range;
wherein the optical property objective further comprises a wavelength range input by the user.

23. The computer-implemented method of claim 19, wherein:
the receiving of the optical property objective comprises receiving a response of maximizing an area under a reflectance curve ratio; and
the method further comprises:
in response to the optical property objective comprising maximizing an area under a reflectance curve ratio, prompting a user to input a wavelength range;
wherein the optical property objective further comprises a wavelength range input by the user.

24. The computer-implemented method of claim 19, wherein the displaying the request for the decision variable comprises presenting an options to vary and hold constant decision variables.

25. The computer-implemented method of claim 19, wherein:
the receiving of the decision variable comprises receiving an indication that a first decision variable should be varied; and
the method further comprises:
in response to receipt of the indication that the first decision variable should be varied, displaying a prompt to enter a decision variable value range.

26. The computer-implemented method of claim 19, wherein:
the receiving of the decision variable comprises receiving an indication that a first decision variable should be held constant; and
the method further comprises:
in response to receipt of the indication that the first decision variable should be held constant, displaying a prompt to enter a single numerical value.

27. The computer-implemented method of claim 19, wherein the decision variable comprises at least one of:
nanoparticle size, nanoparticle absorption, nanoparticle refractive index, volume fraction of the microsphere, microsphere size, refractive index of the bulk medium, concentration of the microspheres, or thickness of the bulk film.

28. The computer-implemented method of claim 19, wherein the decision variable comprises a matrix material, and the method further comprises:
accessing, by the processor, a library of materials to determine at least one matrix material refractive index property of the matrix material.

29. The computer-implemented method of claim 19, wherein the decision variable comprises a matrix material specified by a continuous variable.

30. The computer-implemented method of claim 19, wherein the optimization parameter comprises a differential evolution parameter comprising:
a number of generations;
a population size;
a crossover probability; or
a weighting factor.

31. The computer-implemented method of claim 19, wherein the optimized reflectance curve is displayed, and comprises a graph of a reflectance vs. wavelength.

32. The computer-implemented method of claim 19, wherein the optimized material property is displayed, and comprises at least one of:
a nanoparticle size;
a void size of a nanoparticle material; or
a formulation refractive index of the nanoparticle material.

33. The computer-implemented method of claim 19, wherein the optimized material property is displayed, and comprises at least one of:
nanoparticle absorption, nanoparticle refractive index, volume fraction of the microsphere, microsphere size, refractive index of the bulk medium, concentration of the microspheres, or thickness of the bulk film.

34. The computer-implemented method of claim 19, wherein the decision variable comprises a name of a nanoparticle material.

35. A computer system comprising one or more processors configured to:
display a request for an optical property objective;
receive the optical property objective;
display a request for a decision variable;
receive the decision variable;
display a request for an optimization parameter;
receive the optimization parameter; and
display: (i) an optimized reflectance curve, optimized transmittance curve, or optimized absorption curve, or (ii) an optimized material property, wherein the displayed (i) optimized reflectance curve, or (ii) optimized material property was generated based on the optical property objective, the decision variable, and the optimization parameter.
